(19) United States
(12) Patent Application Publication
(10) Pub. No.: US 2018/0193332 A1
(43) Pub. Date: Jul. 12, 2018

(54) LOW-TEMPERATURE STABLE OPIOID ANTAGONIST SOLUTIONS

(71) Applicant: Adapt Pharma Operations Limited, Dublin (IE)

(72) Inventors: Ryan Loughlin, Dublin (IE); Fintan Keegan, Dublin (IE); Robert Gerard Bell, Clearwater, FL (US)

(73) Assignee: Adapt Pharma Operations Limited

(21) Appl. No.: 15/864,708

(22) Filed: Jan. 8, 2018

Related U.S. Application Data
(60) Provisional application No. 62/569,708, filed on Oct. 9, 2017.

(51) Int. Cl. A61K 31/485; A61K 47/10; A61K 47/18; A61K 47/02; A61K 9/00; A61M 11/00

(52) U.S. Cl. CPC A61K 31/485; A61K 47/10; A61K 47/186; A61M 11/007; A61K 47/02; A61K 9/0073; A61K 47/18

(57) ABSTRACT

The present invention relates to pharmaceutical compositions comprising an opioid antagonist, PG, and an isotonicity agent. The pharmaceutical compositions are stable at temperatures as low as −5° C. or lower. Methods of using the pharmaceutical compositions are also disclosed.

### CROSS-REFERENCE TO RELATED APPLICATIONS

This application claims benefit of Ser. No. 62/569,708, filed on 9 Oct. 2017. The entire disclosure of the application identified in this paragraph is incorporated herein by reference.

### FIELD

The present disclosure describes pharmaceutical compositions and methods for treating disease using the same. Particularly, the present disclosure relates to opioid antagonist compositions and methods for treating opioid overdose.

### BACKGROUND

Opioid overdose can give rise to a number of pathological conditions, including respiratory depression. Respiratory depression can quickly result in organ and brain damage, and death. Therefore, an overdose remedy should be fast acting, and capable of immediate use. Naloxone is an opioid receptor antagonist, able to displace opioids from opioid receptors, and thus reverse an overdose. Naloxone is used as an emergency treatment to reverse opioid overdose.

Naloxone can be used in community or hospital settings. Community settings include use by family members, friends or caregivers, and by first responders such as police, fire and life support officers. The product is stored in people's homes, universities, schools, public buildings, police and fire departments/vehicles, ambulances and, hospitals, amongst other settings, so it can be quickly accessed and used when a person suffers an opioid overdose. Naloxone can be stored for an extended period of time, in a variety of storage conditions, prior to its use, and it is important that naloxone continues to be effective. A nasal spray formulation of naloxone label states that the product should be stored at a controlled room temperature 59° F. to 77° F. (15° C. to 25° C.) with excursions permitted between 4° C. to 40° C. (39° F. to 104° F.). It also states the product should not be frozen. A naloxone solution that remains stable across a wide range of temperatures is highly desirable. Nevertheless, the prior art has not achieved such a naloxone formulation.

U.S. Pat. No. 9,561,177 to Keegan et al. reports naloxone formulations for nasal administration. These formulations contain naloxone as well as ethylenediaminetetraacetic acid (EDTA) and benzalkonium chloride (BZK) to achieve a shelf-stable formulation. These formulations show excellent stability at 4° C., 25° C., and 40° C., but there is no information presented by Keegan et al. as to the stability of these formulations at temperatures of 0° C. or below.

US 2016/0199294 to Amancha et al. reports storage sublingual spray formulations comprising an effective amount of naloxone, a pharmaceutically acceptable salt or a derivative thereof, water, propylene glycol (PG), and ethanol as cosolvents, antioxidant, chelating agent, and a permeation enhancer. Amancha teaches, as a preferred embodiment, a sublingual formulation comprising a cosolvent that is a mixture of PG at about 5% w/w and ethanol at about 50% w/w. These formulations are shown to be resistant to precipitation through three freeze/thaw cycles.

U.S. Pat. No. 9,192,570 to Wyse et al. reports naloxone formulations for nasal administration. These formulations include 1% (w/v) PG, 10 mM EDTA, and citric acid. Some of these formulations also include BZK or benzyl alcohol. Wyse reports that PG at the indicated concentration causes naloxone to degrade in solution, and thus concludes that PG is unacceptable as an excipient in a naloxone solution for nasal administration. Wyse purports to find acceptable stability for three years storage at 25° C. and for six months at 40° C. with the formulations containing methyl parabens or benzyl alcohol, but does not report results with freeze/thaw stability.

U.S. Pat. No. 9,216,175 to Amancha et al. report sublingual buprenorphine sprays that optionally contain naloxone. Amancha reports, as one embodiment, a sublingual spray formulation comprising: buprenorphine, a pharmaceutically acceptable salt thereof or a derivative thereof at an amount of about 0.25% to about 9.5% w/w; naloxone, a pharmaceutically acceptable salt thereof or a derivative thereof at an amount of about 0.005% to about 3% w/w; water as a solvent in an amount of about 27.4% w/w to 39.7% w/w; a cosolvent consisting of a mixture of ethanol in an amount of about 55% w/w and PG in an amount of about 5% w/w; and an antioxidant in an amount from about 0.001% to about 0.2% w/w. Amancha reports that both the naloxone and the buprenorphine in these solutions are stable at 25° C. and 40° C. for up to 3 months.

US 2009/0041687 to Beumer et al. describes use of opioid receptor antagonists for the manufacture of topical compositions for suppression of melanin formation in the human skin. An exemplary skin whitening emulsion comprises pemulen TR-1 0.80%, biotin 0.01%, disodium EDTA 0.10%, D-panthenol 0.20%, Hyasol BT 1.00%, Euxyl K 400 0.20%, NaOH 1.00%, PG 5.00%, epigallocatechin gallate 0.50%, genistein 0.10%, niacinamide 0.50% emblica 0.50%, hydroquinone 0.20%, naloxone 2.00%, citric acid, and water.

Lyapunov et al. (2014) Farmatsiya 8:16-19 report a naloxone nasal spray that employs PG as an anti-bacterial preservative at a concentration of 10% (w/v).

### SUMMARY

Pharmaceutical solutions are described herein. These solutions are useful for treating, inter alia, opioid overdose. In an embodiment, there is provided a solution comprising: at least about 2% (w/v) of an opioid antagonist, between about 2% (w/v) and about 25% (w/v) propylene glycol (PG), and isotonicity agent sufficient to achieve an osmolality between about 300 mOsm and 2500 mOsm; wherein the solution comprises no more than about 2% (w/v) of alcohol, and wherein the solution has a dynamic viscosity less than about 100 cP at 21° C. In certain embodiments, the solutions comprise about 2 to about 20% (w/v) of naloxone (e.g., 2% (w/v), 4% (w/v), 6% (w/v), 8% (w/v), 10% (w/v), etc.). In certain embodiments, the solutions also comprise between about 2% (w/v) and about 25% (w/v) PG, and between about 0.2% (w/v) and 1.8% (w/v) of an isotonicity agent (e.g., NaCl, KCl, CaCl₂, MgCl₂, etc.). In certain embodiments, the solutions will contain no more than about 1% (w/v) alcohol (e.g., ethanol, benzyl alcohol, phenol, etc.). In certain embodiments, the solutions will not be part of an emulsion, such as an oil-in-water emulsion or a water-in-oil emulsion.

Also described herein are nasal spray devices containing the pharmaceutical solutions described above. In an embodiment, there is provided a pre-primed nasal spray device, wherein the device comprises a reservoir, a piston, a swirl chamber, and a spray nozzle; and wherein the reservoir contains a solution comprising: at least about 2% (w/v) of an opioid antagonist; between about 2% (w/v) and about 25% (w/v) PG; between about 0.2% and about 1.8% (w/v) of an isotonicity agent; and no more than about 1% (w/v) of alcohol. In certain embodiments, these devices are configured for single use only. In certain embodiments, the devices deliver two doses (“bi-dose devices”) or more than two doses, either simultaneously, or one after another, as required, to reverse the opioid overdose. In certain embodiments, these devices are pre-primed to deliver a dose or doses of a specific quantity of solution (e.g., 50 μL, 100 μL, 150 μL, 200 μL, etc.). The present disclosure also provides a mist, wherein the mist stands adjacent to a spray nozzle, and wherein the mist comprises droplets of a solution, and wherein no more than about 10% of the droplets have a diameter less than 10 μm as measured by laser diffraction at 3 cm and 6 cm from the spray nozzle, and wherein the solution has a dynamic viscosity less than 100 cP at 21° C., and wherein the solution comprises: at least about 2% (w/v) of an opioid antagonist; between about 2% (w/v) and about 15% (w/v) PG; between about 0.2% and about 1.8% (w/v) of an isotonicity agent; and no more than about 1% (w/v) of alcohol.

Also described herein are methods of using the solutions and devices described above. In an embodiment, there is provided a method of treating opioid overdose in a patient in need thereof, the method comprising: delivering a spray from a pre-primed, single-use nasal spray device into a nostril of the patient, wherein a reservoir of the device contains a pharmaceutical solution comprising at least about 2% (w/v) of an opioid antagonist, and between about 2% (w/v) and about 15% (w/v) PG. In certain embodiments, these methods can be used to treat opioid overdose in a patient, e.g., an unconscious patient or a patient experiencing respiratory depression. In certain embodiments, these methods involve delivering a specific quantity of solution (e.g., 50 μL, 100 μL, 200 μL, etc.) from the devices described into the nostril of an overdose patient. In certain embodiments, only one delivery of liquid is necessary, while in other methods, two or more deliveries are required to reverse the effects of the opioid overdose. In certain embodiments, the device delivers two doses (“bi-dose devices”), or more than two doses, either simultaneously, or one after another, as required, to treat opioid overdose in a patient in need thereof.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

FIG. 1 shows temperature trends over time, highlighting the freezing points of media (with naloxone HCl) containing various amounts of PG (0%, 10%, 20%, and 30% w/v) with agitation at 700 RPM.

FIG. 2 shows a solubility curve for naloxone HCl without cosolvent (0.2% w/v EDTA-Na₂). Calculation based on a standard purity of 100% and a theoretical water content of 9.01%.

FIG. 3 shows a solubility curve for naloxone HCl in 20% w/v propylene glycol (0.2% w/v EDTA-Na₂).

FIG. 4 shows a solubility curve for naloxone HCl in 20% w/v propylene glycol (0.2%, 0.1%, and 0% w/v EDTA-Na₂).

FIG. 5 shows a solubility curve for naloxone HCl in 15% w/v propylene glycol (0.2%, 0.1%, 0.5%, and 0% w/v EDTA-Na₂).

FIG. 6 shows a solubility for naloxone HCl in 20% w/v propylene glycol (0.1% w/v EDTA-Na₂) with and without 4% (w/v) sorbitol.

FIG. 7 shows an RP-HPLC chromatograph taken prior to storage.

FIG. 8 shows an RP-HPLC chromatograph taken after 6 days of storage at 50° C.

FIG. 9 shows an RP-HPLC chromatograph taken after 10 days of storage at 50° C.

### DETAILED DESCRIPTION

The following description is merely exemplary in nature and is not intended to limit the present disclosure, application or uses.

### A. Definitions

As used herein, “low temperature stability” refers to the ability of a solution to tolerate storage at temperatures to approximately −10° C. without losing essential elements of the solution. “Low temperature stability” also conveys that if the solution should freeze, it can be thawed without compromising potency or stability.

As used herein, a “caregiver” is any person who administers naloxone to a patient suspected of suffering from opioid overdose. By way of non-limiting example, a caregiver can be a friend, a family member, a first responder, a physician or nurse, or a stranger whose only connection to the patient is to have noticed the patient in a state of distress (e.g., respiratory depression).

As used herein, an “opioid antagonist” is a compound that counteracts the effects of opioid binding to an opioid receptor. Non-limiting examples of opioid antagonists include naloxone, or a pharmaceutically acceptable salt and/or solvate thereof (e.g., naloxone HCl or naloxone HCl.2H₂O). Additional non-limiting examples include naltrexone, nalmefene, diprenorphine, nalorphine, nalorphine dinicotinate, levallorphan, samidorphan, and nalodeine.

As used herein, an “isotonicity agent” is an additive that is added to a solution to bring its tonicity into an isotonic balance with the human nasal mucosa. Non-limiting examples of isotonicity agents include NaCl, KCl, CaCl₂, MgCl₂, NaBr, KBr, CaBr₂, MgBr₂, dextrose, glycerin, and mannitol.

As used herein, a “stabilizing agent” is an additive that is added to a solution to prevent the degradation of another agent. Non-limiting examples of stabilizing agents include calcium, sodium, and disodium ethylenediaminetetraacetic acid (EDTA), ethylene glycol-bis(β-aminoethyl ether)-N,N,N′,N′-tetraacetic acid (EGTA), sorbitol, dimercaptosuccinic acid (DMSA), calcium versetamide Na, calteridol, and diethylenetriaminepentaacetic acid (DTPA).

As used herein, a “preservative” is an additive that is added to a solution to prevent the growth of a biological contaminant (e.g., bacteria or fungus), or to prevent the chemical degradation of components of the solution. Non-limiting examples of preservatives include quaternary ammonium compounds (e.g., benzalkonium chloride, abbreviated “BZK”), alkyl parabens, citric acid, and alcohols. As used herein, “preservative” does not include glycols (e.g., propylene glycol and polyethylene glycol).

As used hererin, an “alcohol” is an organic molecule comprised of an alkyl, aryl, or arylalkyl backbone substituted with a single hydroxyl moiety. Non-limiting examples of alcohols include benzyl alcohol, phenylethyl alcohol, chlorobutanol, butylated hydroxytoluene, butylated hydroxyanisole, and ethanol. As used herein, “alcohol” does not include glycols.

As used herein, a “cosolvent” is a liquid that is added to a mixture of water and another substance. Non-limiting examples of cosolvents include benzyl benzoate, N,N-dimethylacetamide, glycerol, vegetable oil (e.g., poppyseed oil, peanut oil, soy oil, safflower oil, castor oil, cottonseed oil, sesame oil, and sunflower oil), alcohols (e.g., ethanol), surfactants (e.g., Solutol®), and diols (e.g., propylene glycol and polyethylene glycols such as PEG 400 and PEG 3500).

The term “fentanyl derivative” as used herein refers to a molecule of Formula (I)

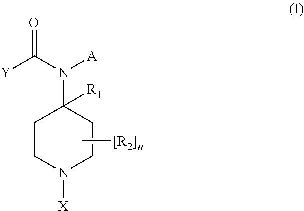

wherein A is aryl or heteroaryl optionally substituted with halo, C₁-C₃ alkyl, or C₁-C₃ alkoxy,

X is C₁-C₃ alkyl or hydroxyethyl, optionally substituted with —COOCH₃, aryl, or heteroaryl optionally substituted with both C₁-C₃ alkyl and ═O,

Y is C₁-C₄ alkyl, C₂-C₃ alkenyl, C₁-C₃ alkoxy, C₁-C₃ alkoxyalkyl, cycloalkyl, or heteroaryl,

R₁ and R₂ are each independently selected from the group consisting of phenyl, C₁-C₃ alkyl, C₂-C₃ alkenyl, C₁-C₃ alkoxyalkyl, or C₁-C₃ alkoxy, and —COOCH₃, and

n is 1, 2, or 3.

Non-limiting examples of fentanyl derivatives are disclosed in WO 2017/049181 to Keegan et al.

The term “titrate” as used herein with reference to opioid receptors conveys a process by which naloxone is administered step-wise in small doses until opioid drug has been displaced from just enough receptors to reverse an overdose while the user retains a large enough percentage of receptors occupied by opioids to sustain an analgesic effect. As used herein, “titrate” does not refer to the titration of naloxone by medical professionals who appropriately administer additional doses of naloxone if the initial dose(s) do(es) not achieve a sufficient reversal of an opioid overdose.

All mentioned documents are incorporated by reference as if herein written. When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations.

### B. Pharmaceutical Solutions

Solutions described herein comprise an opioid antagonist as an active ingredient dissolved in a suitable medium. In an embodiment, the opioid antagonist is selected from naloxone, naltrexone, nalmefene, diprenorphine, nalorphine, nalorphine dinicotinate, levallorphan, samidorphan, nalodeine, a pharmaceutically acceptable salt thereof, a solvate thereof and a mixture thereof. In certain embodiments, naltrexone is used at a dose between about 5 mg and 50 mg, for example about 10 mg, about 25 mg, or about 35 mg. In certain embodiments, nalmefene is used at a dose between about 1 mg and about 20 mg, for example about 18 mg. In certain embodiments, diprenorphine is used at a dose between about 1 mg and about 5 mg, for example about 2 mg, about 3 mg, or about 4 mg. In certain embodiments, levallorphan is used at a dose between about 1 mg and about 5 mg.

In certain embodiments, naloxone is provided as free base. In certain embodiments, naloxone is provided as a salt (e.g., a hydrochloride or an acetate salt). In certain embodiments, naloxone is provided as a solvate, or a solvate of a salt (e.g., naloxone hydrochloride dihydrate). Regardless of how naloxone is provided, the ultimate naloxone solution comprises between about 1% (w/v) and about 15% (w/v) of naloxone, for example between about 2% (w/v) and about 12% (w/v), or between about 3% (w/v) and about 10% (w/v). In certain embodiments, the solution comprises about 2% (w/v), about 3% (w/v), about 4% (w/v), about 5% (w/v), about 6% (w/v), about 7% (w/v), about 8% (w/v), about 9% (w/v), about 10% (w/v), about 11% (w/v), or about 12% (w/v) of naloxone or naloxone HCl. In some other embodiments, the solution comprises about 2% (w/v), about 4% (w/v), about 6% (w/v), about 8% (w/v), or about 10% (w/v) of naloxone or naloxone HCl.

In certain embodiments, the naloxone solution described herein may also comprise a cosolvent. In certain embodiments, the cosolvent is selected from the group consisting of benzyl benzoate, N,N-dimethylacetamide, glycerol, vegetable oil, ethanol, propylene glycol, and combinations thereof. In certain embodiments, for example, the solutions may comprise between about 3% (w/v) and about 25% (w/v) PG, for example between about 5% (w/v) and about 15% (w/v) or between about 5% (w/v) and 10% (w/v). In certain embodiments, the solutions may contain about 5% (w/v), about 6% (w/v), about 7% (w/v), about 8% (w/v), about 9% (w/v), about 10% (w/v), about 11% (w/v), about 12% (w/v), about 15% (w/v), about 20% (w/v), about 22% (w/v), about 24% (w/v), and about 25% (w/v) of PG. In some embodiments, the solution comprises about 5% (w/v), about 6% (w/v), or about 7% (w/v) of PG. In some other embodiments, the solution comprises about 9% (w/v), about 10% (w/v), or about 11% (w/v) of PG. In certain embodiments, the solutions will not be part of an emulsion, such as an oil-in-water emulsion or a water-in-oil emulsion. In certain embodiments, the solutions will not contain any detectable emulsifiers or emulsifying agents. Common emulsifying agents include (but are not limited to) calcium stearoyl di-lactate (CSL), polyglycerol ester (PGE), sorbitan ester (SOE), and propylene glycol monoester (PGME).

In certain embodiments, the solutions may optionally comprise alcohol, for example, between about 5% (w/v) and about 15% (w/v) ethanol. In certain other embodiments, the solutions may comprise no more than about 10% (w/v) (for example, no more than about 9%, no more than about 8%, no more than about 7%, no more than about 6%, no more than about 5%, no more than about 4%, no more than about 3%, no more than about 2%, or no more than about 1% w/v) of alcohol, and in particular no more than about 10% (w/v) (for example, no more than about 9%, no more than about 8%, no more than about 7%, no more than about 6%, no more than about 5%, no more than about 4%, no more than about 3%, no more than about 2%, or no more than about 1% w/v) of ethanol. In certain embodiments, the solutions contain no detectable amount of alcohol (in particular, no detectable amount of ethanol).

In certain embodiments, the naloxone solution described herein may also comprise an isotonicity agent, optionally in combination with the cosolvent described above. In certain embodiments, the isotonicity agent is selected from the group consisting of dextrose, mannitol, amino acids, cyclodextrin, glucose, inositol, lactose, sorbitol, sucrose, trehalose, maltose, MgSO₄, Na₂SO₄, NaCl, KCl, CaCl₂, MgCl₂, NaBr, KBr, CaBr₂, MgBr₂, and combinations thereof. In certain embodiments, the solution contains a quantity of isotonicity agent sufficient to achieve an osmolality between about 300 mOsm and about 2500 mOsm, for example between about 750 mOsm and about 2.5 Osm, between about 1 Osm and about 2.5 Osm, or between about 2.0 Osm and about 2.2 Osm. In certain embodiments, for example, the solutions may comprise between about 0.1% (w/v) and about 2% (w/v) of the isotonicity agent (e.g., NaCl), for example between about 0.2% (w/v) and about 1.9% (w/v), or between about 0.6% (w/v) and about 1% (w/v). In certain embodiments, the solutions may contain about 0.1% (w/v), about 0.2% (w/v), about 0.3% (w/v), about 0.4% (w/v), about 0.5% (w/v), about 0.6% (w/v), about 0.61% (w/v), about 0.62% (w/v), about 0.63% (w/v), about 0.64% (w/v), about 0.65% (w/v), about 0.66% (w/v), % (w/v), about 0.67% (w/v), about 0.68% (w/v), about 0.69% (w/v), about 0.7% (w/v), about 0.71% (w/v), about 0.72% (w/v), about 0.73% (w/v), about 0.74% (w/v), about 0.75% (w/v), about 0.76% (w/v), about 0.77% (w/v), about 0.78% (w/v), about 0.79% (w/v), and about 0.8% (w/v) of the isotonicity agent. In certain embodiments, the solutions may comprise between about 0.5% (w/v) and about 1.9% (w/v) NaCl.

In certain embodiments, the naloxone solution described herein may also comprise a stabilizing agent, optionally in combination with the cosolvent and/or isotonicity agent described above. In certain embodiments, the stabilizing agent is selected from the group consisting of calcium, sodium, and disodium EDTA, EGTA, sorbitol, DMSA, calcium versetamide Na, calteridol, DTPA, pentetic acid, and combinations thereof. In certain embodiments, for example, the solutions may comprise between about 0.05% and about 0.5% (w/v) of the stabilizing agent (e.g., EDTA), for example between about 0.1% (w/v) and about 0.3% (w/v), or between about 0.15% (w/v) and about 0.25% (w/v). In certain embodiments, the solutions may contain about 0.05% (w/v), about 0.1% (w/v), about 0.15% (w/v), about 0.2% (w/v), about 0.25% (w/v), about 0.3% (w/v), about 0.35% (w/v), about 0.4% (w/v), about 0.45% (w/v), about 0.5% (w/v), and about 0.55% (w/v) of the stabilizing agent (e.g., EDTA). In certain embodiments, the solutions may comprise about 0.1% (w/v) EDTA, EGTA, or disodium edetate. In certain embodiments, the solution may comprise up to about 5% (w/v) sorbitol, or up to about 4.5% (w/v) sorbitol.

In certain embodiments, the naloxone solution described herein may also comprise a preservative, optionally in combination with the cosolvent and/or stabilizing agent and/or isotonicity agent described above. In certain embodiments the preservative is selected from the group consisting of BZK, alkyl parabens, citric acid, propylene glycol, benzyl alcohol, ethanol, and combinations thereof. In certain embodiments, for example, the solutions may comprise between about 0.005% and about 0.119% (w/v) of the preservative (e.g., BZK), for example between about 0.005% and about 0.015% (w/v), or between about 0.01% (w/v) and about 0.02% (w/v). In certain embodiments, the solutions may contain about 0.005% (w/v), about 0.01% (w/v), about 0.015% (w/v), about 0.02% (w/v), about 0.025% (w/v), about 0.03% (w/v), about 0.035% (w/v), about 0.04% (w/v), about 0.045% (w/v), about 0.05% (w/v), about 0.055% (w/v), about 0.06% (w/v), about 0.1% (w/v), and about 0.2% (w/v) of the preservative (e.g., BZK). Additionally or alternatively, the solution may contain no more than about 0.09% (w/v) (for example, no more than about 0.08% (w/v), no more than about 0.07% (w/v), no more than about 0.06% (w/v), no more than about 0.05% (w/v), no more than about 0.04% (w/v), no more than about 0.03% (w/v), no more than about 0.02% (w/v), no more than about 0.01% (w/v), no more than about 0.005% (w/v), or no more than about 0.001% (w/v) of BZK, alkyl paraben, citric acid, and/or benzyl alcohol. In certain embodiments, the solution may contain no detectable amount of BZK, alkyl paraben, citric acid, and/or benzyl alcohol.

The solutions described herein can comprise all of the ingredients described above, or only some of them. For example, in certain embodiments, the solutions comprise naloxone, water, a cosolvent, an isotonicity agent, and optionally an acid. In certain embodiments, the solutions consist of naloxone, water, a cosolvent, an isotonicity agent, and optionally an acid. In certain embodiments the solutions consist of naloxone, water, a cosolvent, an isotonicity agent, a stabilizing agent, and optionally an acid or buffer. In certain embodiments, the solutions consist of naloxone, water, a cosolvent, an isotonicity agent, a preservative, and optionally an acid. In certain embodiments, the solutions consist of naloxone, water, a cosolvent, an isotonicity agent, a stabilizing agent, a preservative, and optionally an acid, base, or buffer.

The pH and osmolality of the solutions described herein must be appropriate to ensure that the naloxone delivered into the nasal cavity can be absorbed into the blood. The solutions described herein will have a pH between about 3 and about 7, for example between about 3.5 and about 5.5. Where the pH of the solution lies outside of the desired range once all of the ingredients are dissolved in the solution, an appropriate quantity of acid, base, or buffer can be added as necessary to adjust the pH before bringing the solution to its final volume. In certain embodiments, the acid is an inorganic acid. In certain embodiments, the acid is HCl or H₂SO₄. In certain embodiments the base is NaOH or KOH. In certain embodiments the buffer is phosphate buffer, acetate buffer, potassium hydrogen phthalate buffer, glycine buffer, disodium hydrogen phthalate buffer, sodium dihydrogen orthophosphate buffer, carbonate buffer, benzoate buffer, hydrobromic acid buffer, lactic acid buffer, tartaric acid buffer, or citrate buffer.

In certain embodiments, the naloxone solutions described herein may also comprise impurities. As used herein, an “impurity” is any detectable chemical species whose presence in the solution was not intended by the manufacturer. An “impurity” may be noxious, but it may also be innocuous, and the detection of an impurity does not necessarily indicate that the solution is toxic or otherwise unsuitable for administration to a patient. In certain embodiments, the incidence of impurities increases during storage of the solutions. Certain impurities can be detected by reverse-phase high pressure liquid chromatography (RP-HPLC) that have relative retention times of less than one. For example, in certain embodiments after 6 and/or 10 days of storage the formulations disclosed herein may show impurities with retention times of about 3.9, 5.7, 6.5, and 20 minutes when measured by by reverse-phase high pressure liquid chromatography (RP-HPLC) as described below. Certain impurities can also be detected by RP-HPLC that have relative retention times greater than one. The RP-HPLC method utilizes a gradient mobile phase (25 mM sodium phosphate at pH 6.8:acetonitrile), with a flow rate at 0.8 mL/min, and ultra-violet (UV) detection at 229 nm, 7.5 μL injection volume, and a C6-phenyl column with a column temperature of 50° C. for a run time of 25 minutes.

In certain embodiments, the osmolality of the solutions will be no less than about 500 mOsm, for example at least about 600 mOsm, at least about 700 mOsm, at least about 800 mOsm, at least about 900 mOsm, at least about 1 Osm, at least about 1.1 Osm, at least about 1.25 Osm, at least about 1.5 Osm, at least about 1.75 Osm, at least about 2 Osm, or at least about 2.1 Osm. Where the osmolality is too low, it can be adjusted by dissolving additional solutes as necessary to bring the osmolality within the desired range.

The viscosity of the solutions must also be appropriate to be sprayed into a fine mist for delivery into the nasal cavities of a human or other animal (e.g., a dog, for example a service dog or police dog). A solution that is too viscous can occlude the spray nozzle and frustrate timely and complete delivery. The solutions described herein should have a dynamic viscosity less than about 100 cP at 21° C., for example less than about 90 cP, less than about 80 cP, less than about 70 cP, less than about 60 cP, less than about 50 cP, less than about 10 cP, less than about 5 cP, less than about 2 cP, less than about 1.8 cP, less than about 1.6 cP, less than about 1.4 cP, less than about 1.2 cP, or about 1 cP at 21° C. In certain embodiments, the solutions will have a dynamic viscosity that is greater than about 0.2 cP at 21° C. (for example greater than about 0.4 cP, greater than about 0.6 cP, greater than about 0.8 cP, greater than 1 cP, greater than about 2 mP, greater than about 10 cP, or greater than about 50 cP at 21° C.), but still less than 100 cP at 21° C. In certain embodiments, the solutions will have a dynamic viscosity of about 2.0 cP, about 2.1 cP, about 2.2 cP, about 2.3 cP, about 2.4 cP, or about 2.5 cP at 21° C.

Depending on the concentration of the solution, one will need to administer different amounts of the solution to reverse opioid overdose per spray. In certain embodiments a single spray will be enough, while in other embodiments more than one spray may be required. For example, in certain embodiments, it will be necessary to administer, per spray, between about 80 μL and about 150 μL, (e.g., about 90 μL, about 100 μL, about 110 μL, about 120 μL, about 130 μL, or about 140 μL) of a 2% (w/v) naloxone HCl solution, a 3% (w/v) naloxone HCl solution, a 4% (w/v) naloxone HCl solution, a 5% (w/v) naloxone HCl solution, a 6% (w/v) naloxone HCl solution, a 7% (w/v) naloxone HCl solution, an 8% (w/v) naloxone HCl solution, a 9% (w/v) naloxone HCl solution, or a 10% (w/v) naloxone HCl solution, depending on the strength and concentration of the opioid(s) that has triggered the overdose. As the concentration of naloxone increases or decreases, the volume that must be delivered can be increased or decreased accordingly to achieve a given quantity of naloxone delivered.

In certain embodiments, the naloxone solutions described herein have a lower freezing point, or enhanced stability at lower temperatures than the currently available Narcan Nasal Spray product. This enhanced stability at lower temperatures and/or lower freezing point may enable storage at low temperatures for extended periods of time or use in temperatures or climatic conditions different from the currently available Narcan Nasal Spray product. In certain embodiments, the solutions described herein maintain solubility of naloxone through multiple free/thaw cycles. In certain embodiments, the solutions have a freezing point of −5° C. to −20° C., for example no more than about −5° C., no more than about −6° C., no more than about −7° C., no more than about −8° C., no more than about −9° C., no more than about −10° C., no more than about −11° C., no more than about −12° C., no more than about −13° C., no more than about −14° C., no more than about −15° C., no more than about −16° C., no more than about −17° C., no more than about −18° C., no more than about −19° C., or no more than about −20° C.

In certain embodiments, the solutions described herein exist as a mist, for example a mist adjacent to the nozzle of a spray device. Spray characterization (e.g., plume geometry, spray pattern, pump delivery, droplet size distribution, DSD) of the mist may be measured under specified experimental and instrumental conditions by appropriate and validated and/or calibrated analytical procedures known in the art. These include photography, laser diffraction, and impaction systems (cascade impaction, next generation impaction (NGI), etc.). Droplet size distribution can be controlled in terms of ranges for the D10, D50, D90, span [(D90-D10)/D50], and percentage of droplets less than 10 mm. The particle diameter “(D)” designations refer to the representative diameter where 10% (D10), 50% (D50) and 90% (D90) of the total volume of the liquid sprayed is made up of droplets with diameters smaller than or equal to the stated value.

In certain embodiments, these mists comprise droplets of naloxone solution, wherein no more than about 10%, for example no more than about 5%, of the droplets have a diameter less than 10 μm. In certain embodiments, the percent of droplets less than 10 μm will be less than about 2%. In certain embodiments, the percent of droplets less than 10 μm will be less than about 1%. In certain embodiments, the prevalent median droplet size is between about 30 and about 100 μm. In certain embodiments, the formulation will have a Dv(50) of 30-70 μm and a Dv(90)<100 μm.

In an embodiment, a solution has a volume of about 80 μL to about 200 μL, for example about 150 μL, about 100 μL, or about 80 μL. All solutions contain buffer (e.g., phosphate buffer) or acid (e.g., hydrochloric acid) sufficient to achieve a pH of 3.5-5.5. Non-limiting examples of such solutions are set forth in Table 1 below.

TABLE 1
Exemplary aqueous naloxone formulations, pH 3.5-5.5 (all ingredient
numbers show % w/v) and 0% sorbitol

| | | | Na₂ | | |
|---|---|---|---|---|---|
| Ex. # | Nlxn.* | PG | EDTA | NaCl | BZK |
| 1 | 2 | 20 | 0.25 | 0.74 | 0.01 |
| 2 | 2 | 15 | 0.25 | 0.74 | 0.01 |
| 3 | 2 | 10 | 0.25 | 0.74 | 0.01 |
| 4 | 2 | 5 | 0.25 | 0.74 | 0.01 |
| 5 | 2 | 20 | 0.20 | 0.74 | 0.01 |
| 6 | 2 | 15 | 0.20 | 0.74 | 0.01 |
| 7 | 2 | 10 | 0.20 | 0.74 | 0.01 |
| 8 | 2 | 5 | 0.20 | 0.74 | 0.01 |
| 9 | 2 | 20 | 0.15 | 0.74 | 0.01 |
| 10 | 2 | 15 | 0.15 | 0.74 | 0.01 |
| 11 | 2 | 10 | 0.15 | 0.74 | 0.01 |
| 12 | 2 | 5 | 0.15 | 0.74 | 0.01 |
| 13 | 2 | 20 | 0.10 | 0.74 | 0.01 |
| 14 | 2 | 15 | 0.10 | 0.74 | 0.01 |
| 15 | 2 | 10 | 0.10 | 0.74 | 0.01 |
| 16 | 2 | 5 | 0.10 | 0.74 | 0.01 |
| 17 | 2 | 20 | 0.25 | 0.74 | 0 |
| 18 | 2 | 15 | 0.25 | 0.74 | 0 |
| 19 | 2 | 10 | 0.25 | 0.74 | 0 |
| 20 | 2 | 5 | 0.25 | 0.74 | 0 |
| 21 | 2 | 20 | 0.20 | 0.74 | 0 |
| 22 | 2 | 15 | 0.20 | 0.74 | 0 |
| 23 | 2 | 10 | 0.20 | 0.74 | 0 |
| 24 | 2 | 5 | 0.20 | 0.74 | 0 |
| 25 | 2 | 20 | 0.15 | 0.74 | 0 |
| 26 | 2 | 15 | 0.15 | 0.74 | 0 |
| 27 | 2 | 10 | 0.15 | 0.74 | 0 |
| 28 | 2 | 5 | 0.15 | 0.74 | 0 |
| 29 | 2 | 20 | 0.10 | 0.74 | 0 |
| 30 | 2 | 15 | 0.10 | 0.74 | 0 |
| 31 | 2 | 10 | 0.10 | 0.74 | 0 |
| 32 | 2 | 5 | 0.10 | 0.74 | 0 |
| 33 | 2 | 20 | 0.25 | 0.64 | 0.01 |
| 34 | 2 | 15 | 0.25 | 0.64 | 0.01 |
| 35 | 2 | 10 | 0.25 | 0.64 | 0.01 |
| 36 | 2 | 5 | 0.25 | 0.64 | 0.01 |
| 37 | 2 | 20 | 0.20 | 0.64 | 0.01 |
| 38 | 2 | 15 | 0.20 | 0.64 | 0.01 |
| 39 | 2 | 10 | 0.20 | 0.64 | 0.01 |
| 40 | 2 | 5 | 0.20 | 0.64 | 0.01 |
| 41 | 2 | 20 | 0.15 | 0.64 | 0.01 |
| 42 | 2 | 15 | 0.15 | 0.64 | 0.01 |
| 43 | 2 | 10 | 0.15 | 0.64 | 0.01 |
| 44 | 2 | 5 | 0.15 | 0.64 | 0.01 |
| 45 | 2 | 20 | 0.10 | 0.64 | 0.01 |
| 46 | 2 | 15 | 0.10 | 0.64 | 0.01 |
| 47 | 2 | 10 | 0.10 | 0.64 | 0.01 |
| 48 | 2 | 5 | 0.10 | 0.64 | 0.01 |
| 49 | 2 | 20 | 0.25 | 0.64 | 0 |
| 50 | 2 | 15 | 0.25 | 0.64 | 0 |
| 51 | 2 | 10 | 0.25 | 0.64 | 0 |
| 52 | 2 | 5 | 0.25 | 0.64 | 0 |
| 53 | 2 | 20 | 0.20 | 0.64 | 0 |
| 54 | 2 | 15 | 0.20 | 0.64 | 0 |
| 55 | 2 | 10 | 0.20 | 0.64 | 0 |
| 56 | 2 | 5 | 0.20 | 0.64 | 0 |
| 57 | 2 | 20 | 0.15 | 0.64 | 0 |
| 58 | 2 | 15 | 0.15 | 0.64 | 0 |
| 59 | 2 | 10 | 0.15 | 0.64 | 0 |
| 60 | 2 | 5 | 0.15 | 0.64 | 0 |
| 61 | 2 | 20 | 0.10 | 0.64 | 0 |
| 62 | 2 | 15 | 0.10 | 0.64 | 0 |
| 63 | 2 | 10 | 0.10 | 0.64 | 0 |
| 64 | 2 | 5 | 0.10 | 0.64 | 0 |
| 65 | 4 | 20 | 0.25 | 0.74 | 0.01 |
| 66 | 4 | 15 | 0.25 | 0.74 | 0.01 |
| 67 | 4 | 10 | 0.25 | 0.74 | 0.01 |
| 68 | 4 | 5 | 0.25 | 0.74 | 0.01 |
| 69 | 4 | 20 | 0.20 | 0.74 | 0.01 |
| 70 | 4 | 15 | 0.20 | 0.74 | 0.01 |
| 71 | 4 | 10 | 0.20 | 0.74 | 0.01 |
| 72 | 4 | 5 | 0.20 | 0.74 | 0.01 |
| 73 | 4 | 20 | 0.15 | 0.74 | 0.01 |
| 74 | 4 | 15 | 0.15 | 0.74 | 0.01 |
| 75 | 4 | 10 | 0.15 | 0.74 | 0.01 |
| 76 | 4 | 5 | 0.15 | 0.74 | 0.01 |
| 77 | 4 | 20 | 0.10 | 0.74 | 0.01 |
| 78 | 4 | 15 | 0.10 | 0.74 | 0.01 |
| 79 | 4 | 10 | 0.10 | 0.74 | 0.01 |
| 80 | 4 | 5 | 0.10 | 0.74 | 0.01 |
| 81 | 4 | 20 | 0.25 | 0.74 | 0 |
| 82 | 4 | 15 | 0.25 | 0.74 | 0 |
| 83 | 4 | 10 | 0.25 | 0.74 | 0 |
| 84 | 4 | 5 | 0.25 | 0.74 | 0 |
| 85 | 4 | 20 | 0.20 | 0.74 | 0 |
| 86 | 4 | 15 | 0.20 | 0.74 | 0 |
| 87 | 4 | 10 | 0.20 | 0.74 | 0 |
| 88 | 4 | 5 | 0.20 | 0.74 | 0 |
| 89 | 4 | 20 | 0.15 | 0.74 | 0 |
| 90 | 4 | 15 | 0.15 | 0.74 | 0 |
| 91 | 4 | 10 | 0.15 | 0.74 | 0 |
| 92 | 4 | 5 | 0.15 | 0.74 | 0 |
| 93 | 4 | 20 | 0.10 | 0.74 | 0 |
| 94 | 4 | 15 | 0.10 | 0.74 | 0 |
| 95 | 4 | 10 | 0.10 | 0.74 | 0 |
| 96 | 4 | 5 | 0.10 | 0.74 | 0 |
| 97 | 4 | 20 | 0.25 | 0.64 | 0.01 |
| 98 | 4 | 15 | 0.25 | 0.64 | 0.01 |
| 99 | 4 | 10 | 0.25 | 0.64 | 0.01 |
| 100 | 4 | 5 | 0.25 | 0.64 | 0.01 |
| 101 | 4 | 20 | 0.20 | 0.64 | 0.01 |
| 102 | 4 | 15 | 0.20 | 0.64 | 0.01 |
| 103 | 4 | 10 | 0.20 | 0.64 | 0.01 |
| 104 | 4 | 5 | 0.20 | 0.64 | 0.01 |
| 105 | 4 | 20 | 0.15 | 0.64 | 0.01 |
| 106 | 4 | 15 | 0.15 | 0.64 | 0.01 |
| 107 | 4 | 10 | 0.15 | 0.64 | 0.01 |
| 108 | 4 | 5 | 0.15 | 0.64 | 0.01 |
| 109 | 4 | 20 | 0.10 | 0.64 | 0.01 |
| 110 | 4 | 15 | 0.10 | 0.64 | 0.01 |
| 111 | 4 | 10 | 0.10 | 0.64 | 0.01 |
| 112 | 4 | 5 | 0.10 | 0.64 | 0.01 |
| 113 | 4 | 20 | 0.25 | 0.64 | 0 |
| 114 | 4 | 15 | 0.25 | 0.64 | 0 |
| 115 | 4 | 10 | 0.25 | 0.64 | 0 |
| 116 | 4 | 5 | 0.25 | 0.64 | 0 |
| 117 | 4 | 20 | 0.20 | 0.64 | 0 |
| 118 | 4 | 15 | 0.20 | 0.64 | 0 |
| 119 | 4 | 10 | 0.20 | 0.64 | 0 |
| 120 | 4 | 5 | 0.20 | 0.64 | 0 |
| 121 | 4 | 20 | 0.15 | 0.64 | 0 |
| 122 | 4 | 15 | 0.15 | 0.64 | 0 |
| 123 | 4 | 10 | 0.15 | 0.64 | 0 |
| 124 | 4 | 5 | 0.15 | 0.64 | 0 |
| 125 | 4 | 20 | 0.10 | 0.64 | 0 |
| 126 | 4 | 15 | 0.10 | 0.64 | 0 |
| 127 | 4 | 10 | 0.10 | 0.64 | 0 |
| 128 | 4 | 5 | 0.10 | 0.64 | 0 |
| 129 | 6 | 20 | 0.25 | 0.74 | 0.01 |
| 130 | 6 | 15 | 0.25 | 0.74 | 0.01 |
| 131 | 6 | 10 | 0.25 | 0.74 | 0.01 |
| 132 | 6 | 5 | 0.25 | 0.74 | 0.01 |
| 133 | 6 | 20 | 0.20 | 0.74 | 0.01 |
| 134 | 6 | 15 | 0.20 | 0.74 | 0.01 |
| 135 | 6 | 10 | 0.20 | 0.74 | 0.01 |
| 136 | 6 | 5 | 0.20 | 0.74 | 0.01 |
| 137 | 6 | 20 | 0.15 | 0.74 | 0.01 |
| 138 | 6 | 15 | 0.15 | 0.74 | 0.01 |
| 139 | 6 | 10 | 0.15 | 0.74 | 0.01 |
| 140 | 6 | 5 | 0.15 | 0.74 | 0.01 |
| 141 | 6 | 20 | 0.10 | 0.74 | 0.01 |
| 142 | 6 | 15 | 0.10 | 0.74 | 0.01 |
| 143 | 6 | 10 | 0.10 | 0.74 | 0.01 |
| 144 | 6 | 5 | 0.10 | 0.74 | 0.01 |
| 145 | 6 | 20 | 0.25 | 0.74 | 0 |
| 146 | 6 | 15 | 0.25 | 0.74 | 0 |
| 147 | 6 | 10 | 0.25 | 0.74 | 0 |
| 148 | 6 | 5 | 0.25 | 0.74 | 0 |
| 149 | 6 | 20 | 0.20 | 0.74 | 0 |
| 150 | 6 | 15 | 0.20 | 0.74 | 0 |
| 151 | 6 | 10 | 0.20 | 0.74 | 0 |
| 152 | 6 | 5 | 0.20 | 0.74 | 0 |
| 153 | 6 | 20 | 0.15 | 0.74 | 0 |
| 154 | 6 | 15 | 0.15 | 0.74 | 0 |
| 155 | 6 | 10 | 0.15 | 0.74 | 0 |
| 156 | 6 | 5 | 0.15 | 0.74 | 0 |
| 157 | 6 | 20 | 0.10 | 0.74 | 0 |
| 158 | 6 | 15 | 0.10 | 0.74 | 0 |
| 159 | 6 | 10 | 0.10 | 0.74 | 0 |
| 160 | 6 | 5 | 0.10 | 0.74 | 0 |
| 161 | 6 | 20 | 0.25 | 0.64 | 0.01 |
| 162 | 6 | 15 | 0.25 | 0.64 | 0.01 |
| 163 | 6 | 10 | 0.25 | 0.64 | 0.01 |
| 164 | 6 | 5 | 0.25 | 0.64 | 0.01 |
| 165 | 6 | 20 | 0.20 | 0.64 | 0.01 |
| 166 | 6 | 15 | 0.20 | 0.64 | 0.01 |
| 167 | 6 | 10 | 0.20 | 0.64 | 0.01 |
| 168 | 6 | 5 | 0.20 | 0.64 | 0.01 |
| 169 | 6 | 20 | 0.15 | 0.64 | 0.01 |
| 170 | 6 | 15 | 0.15 | 0.64 | 0.01 |
| 171 | 6 | 10 | 0.15 | 0.64 | 0.01 |
| 172 | 6 | 5 | 0.15 | 0.64 | 0.01 |
| 173 | 6 | 20 | 0.10 | 0.64 | 0.01 |
| 174 | 6 | 15 | 0.10 | 0.64 | 0.01 |
| 175 | 6 | 10 | 0.10 | 0.64 | 0.01 |
| 176 | 6 | 5 | 0.10 | 0.64 | 0.01 |
| 177 | 6 | 20 | 0.25 | 0.64 | 0 |
| 178 | 6 | 15 | 0.25 | 0.64 | 0 |
| 179 | 6 | 10 | 0.25 | 0.64 | 0 |
| 180 | 6 | 5 | 0.25 | 0.64 | 0 |
| 181 | 6 | 20 | 0.20 | 0.64 | 0 |
| 182 | 6 | 15 | 0.20 | 0.64 | 0 |
| 183 | 6 | 10 | 0.20 | 0.64 | 0 |
| 184 | 6 | 5 | 0.20 | 0.64 | 0 |
| 185 | 6 | 20 | 0.15 | 0.64 | 0 |
| 186 | 6 | 15 | 0.15 | 0.64 | 0 |
| 187 | 6 | 10 | 0.15 | 0.64 | 0 |
| 188 | 6 | 5 | 0.15 | 0.64 | 0 |
| 189 | 6 | 20 | 0.10 | 0.64 | 0 |
| 190 | 6 | 15 | 0.10 | 0.64 | 0 |
| 191 | 6 | 10 | 0.10 | 0.64 | 0 |
| 192 | 6 | 5 | 0.10 | 0.64 | 0 |
| 193 | 8 | 20 | 0.25 | 0.74 | 0.01 |
| 194 | 8 | 15 | 0.25 | 0.74 | 0.01 |
| 195 | 8 | 10 | 0.25 | 0.74 | 0.01 |
| 196 | 8 | 5 | 0.25 | 0.74 | 0.01 |
| 197 | 8 | 20 | 0.20 | 0.74 | 0.01 |
| 198 | 8 | 15 | 0.20 | 0.74 | 0.01 |
| 199 | 8 | 10 | 0.20 | 0.74 | 0.01 |
| 200 | 8 | 5 | 0.20 | 0.74 | 0.01 |
| 201 | 8 | 20 | 0.15 | 0.74 | 0.01 |
| 202 | 8 | 15 | 0.15 | 0.74 | 0.01 |
| 203 | 8 | 10 | 0.15 | 0.74 | 0.01 |
| 204 | 8 | 5 | 0.15 | 0.74 | 0.01 |
| 205 | 8 | 20 | 0.10 | 0.74 | 0.01 |
| 206 | 8 | 15 | 0.10 | 0.74 | 0.01 |
| 207 | 8 | 10 | 0.10 | 0.74 | 0.01 |
| 208 | 8 | 5 | 0.10 | 0.74 | 0.01 |
| 209 | 8 | 20 | 0.25 | 0.74 | 0 |
| 210 | 8 | 15 | 0.25 | 0.74 | 0 |
| 211 | 8 | 10 | 0.25 | 0.74 | 0 |
| 212 | 8 | 5 | 0.25 | 0.74 | 0 |
| 213 | 8 | 20 | 0.20 | 0.74 | 0 |
| 214 | 8 | 15 | 0.20 | 0.74 | 0 |
| 215 | 8 | 10 | 0.20 | 0.74 | 0 |
| 216 | 8 | 5 | 0.20 | 0.74 | 0 |
| 217 | 8 | 20 | 0.15 | 0.74 | 0 |
| 218 | 8 | 15 | 0.15 | 0.74 | 0 |
| 219 | 8 | 10 | 0.15 | 0.74 | 0 |
| 220 | 8 | 5 | 0.15 | 0.74 | 0 |
| 221 | 8 | 20 | 0.10 | 0.74 | 0 |
| 222 | 8 | 15 | 0.10 | 0.74 | 0 |
| 223 | 8 | 10 | 0.10 | 0.74 | 0 |
| 224 | 8 | 5 | 0.10 | 0.74 | 0 |
| 225 | 8 | 20 | 0.25 | 0.64 | 0.01 |
| 226 | 8 | 15 | 0.25 | 0.64 | 0.01 |
| 227 | 8 | 10 | 0.25 | 0.64 | 0.01 |
| 228 | 8 | 5 | 0.25 | 0.64 | 0.01 |
| 229 | 8 | 20 | 0.20 | 0.64 | 0.01 |
| 230 | 8 | 15 | 0.20 | 0.64 | 0.01 |
| 231 | 8 | 10 | 0.20 | 0.64 | 0.01 |
| 232 | 8 | 5 | 0.20 | 0.64 | 0.01 |
| 233 | 8 | 20 | 0.15 | 0.64 | 0.01 |
| 234 | 8 | 15 | 0.15 | 0.64 | 0.01 |
| 235 | 8 | 10 | 0.15 | 0.64 | 0.01 |
| 236 | 8 | 5 | 0.15 | 0.64 | 0.01 |
| 237 | 8 | 20 | 0.10 | 0.64 | 0.01 |
| 238 | 8 | 15 | 0.10 | 0.64 | 0.01 |
| 239 | 8 | 10 | 0.10 | 0.64 | 0.01 |
| 240 | 8 | 5 | 0.10 | 0.64 | 0.01 |
| 241 | 8 | 20 | 0.25 | 0.64 | 0 |
| 242 | 8 | 15 | 0.25 | 0.64 | 0 |
| 243 | 8 | 10 | 0.25 | 0.64 | 0 |
| 244 | 8 | 5 | 0.25 | 0.64 | 0 |
| 245 | 8 | 20 | 0.20 | 0.64 | 0 |
| 246 | 8 | 15 | 0.20 | 0.64 | 0 |
| 247 | 8 | 10 | 0.20 | 0.64 | 0 |
| 248 | 8 | 5 | 0.20 | 0.64 | 0 |
| 249 | 8 | 20 | 0.15 | 0.64 | 0 |
| 250 | 8 | 15 | 0.15 | 0.64 | 0 |
| 251 | 8 | 10 | 0.15 | 0.64 | 0 |
| 252 | 8 | 5 | 0.15 | 0.64 | 0 |
| 253 | 8 | 20 | 0.10 | 0.64 | 0 |
| 254 | 8 | 15 | 0.10 | 0.64 | 0 |
| 255 | 8 | 10 | 0.10 | 0.64 | 0 |
| 256 | 8 | 5 | 0.10 | 0.64 | 0 |

*Nlxn. = naloxone HCl

In certain embodiments, the solutions described herein may contain sorbitol. Exemplary sorbitol containing embodiments are disclosed in Table 2 below. Each of the solutions disclosed may contain about 1%, about 2%, about 3%, about 4%, about 4.5%, or about 6% (w/v) sorbitol.

TABLE 2
Exemplary aqueous naloxone formulations, pH 3.5-5.5
(all ingredient numbers show % w/v), and about 1%, about
2%, about 3%, about 4%, about 4.5%, or about 6% (w/v) sorbitol

| | | | Na₂ | | |
|---|---|---|---|---|---|
| Ex. # | Nlxn.* | PG | EDTA | NaCl | BZK |
| 1 | 2 | 20 | 0.25 | 0.74 | 0.01 |
| 2 | 2 | 15 | 0.25 | 0.74 | 0.01 |
| 3 | 2 | 10 | 0.25 | 0.74 | 0.01 |
| 4 | 2 | 5 | 0.25 | 0.74 | 0.01 |
| 5 | 2 | 20 | 0.20 | 0.74 | 0.01 |
| 6 | 2 | 15 | 0.20 | 0.74 | 0.01 |
| 7 | 2 | 10 | 0.20 | 0.74 | 0.01 |
| 8 | 2 | 5 | 0.20 | 0.74 | 0.01 |
| 9 | 2 | 20 | 0.15 | 0.74 | 0.01 |
| 10 | 2 | 15 | 0.15 | 0.74 | 0.01 |
| 11 | 2 | 10 | 0.15 | 0.74 | 0.01 |
| 12 | 2 | 5 | 0.15 | 0.74 | 0.01 |
| 13 | 2 | 20 | 0.10 | 0.74 | 0.01 |
| 14 | 2 | 15 | 0.10 | 0.74 | 0.01 |
| 15 | 2 | 10 | 0.10 | 0.74 | 0.01 |
| 16 | 2 | 5 | 0.10 | 0.74 | 0.01 |
| 17 | 2 | 20 | 0.25 | 0.74 | 0 |
| 18 | 2 | 15 | 0.25 | 0.74 | 0 |
| 19 | 2 | 10 | 0.25 | 0.74 | 0 |
| 20 | 2 | 5 | 0.25 | 0.74 | 0 |
| 21 | 2 | 20 | 0.20 | 0.74 | 0 |
| 22 | 2 | 15 | 0.20 | 0.74 | 0 |
| 23 | 2 | 10 | 0.20 | 0.74 | 0 |
| 24 | 2 | 5 | 0.20 | 0.74 | 0 |
| 25 | 2 | 20 | 0.15 | 0.74 | 0 |
| 26 | 2 | 15 | 0.15 | 0.74 | 0 |
| 27 | 2 | 10 | 0.15 | 0.74 | 0 |
| 28 | 2 | 5 | 0.15 | 0.74 | 0 |
| 29 | 2 | 20 | 0.10 | 0.74 | 0 |
| 30 | 2 | 15 | 0.10 | 0.74 | 0 |
| 31 | 2 | 10 | 0.10 | 0.74 | 0 |
| 32 | 2 | 5 | 0.10 | 0.74 | 0 |
| 33 | 2 | 20 | 0.25 | 0.64 | 0.01 |
| 34 | 2 | 15 | 0.25 | 0.64 | 0.01 |
| 35 | 2 | 10 | 0.25 | 0.64 | 0.01 |
| 36 | 2 | 5 | 0.25 | 0.64 | 0.01 |
| 37 | 2 | 20 | 0.20 | 0.64 | 0.01 |
| 38 | 2 | 15 | 0.20 | 0.64 | 0.01 |
| 39 | 2 | 10 | 0.20 | 0.64 | 0.01 |
| 40 | 2 | 5 | 0.20 | 0.64 | 0.01 |
| 41 | 2 | 20 | 0.15 | 0.64 | 0.01 |
| 42 | 2 | 15 | 0.15 | 0.64 | 0.01 |
| 43 | 2 | 10 | 0.15 | 0.64 | 0.01 |
| 44 | 2 | 5 | 0.15 | 0.64 | 0.01 |
| 45 | 2 | 20 | 0.10 | 0.64 | 0.01 |
| 46 | 2 | 15 | 0.10 | 0.64 | 0.01 |
| 47 | 2 | 10 | 0.10 | 0.64 | 0.01 |
| 48 | 2 | 5 | 0.10 | 0.64 | 0.01 |
| 49 | 2 | 20 | 0.25 | 0.64 | 0 |
| 50 | 2 | 15 | 0.25 | 0.64 | 0 |
| 51 | 2 | 10 | 0.25 | 0.64 | 0 |
| 52 | 2 | 5 | 0.25 | 0.64 | 0 |
| 53 | 2 | 20 | 0.20 | 0.64 | 0 |
| 54 | 2 | 15 | 0.20 | 0.64 | 0 |
| 55 | 2 | 10 | 0.20 | 0.64 | 0 |
| 56 | 2 | 5 | 0.20 | 0.64 | 0 |
| 57 | 2 | 20 | 0.15 | 0.64 | 0 |
| 58 | 2 | 15 | 0.15 | 0.64 | 0 |
| 59 | 2 | 10 | 0.15 | 0.64 | 0 |
| 60 | 2 | 5 | 0.15 | 0.64 | 0 |
| 61 | 2 | 20 | 0.10 | 0.64 | 0 |
| 62 | 2 | 15 | 0.10 | 0.64 | 0 |
| 63 | 2 | 10 | 0.10 | 0.64 | 0 |
| 64 | 2 | 5 | 0.10 | 0.64 | 0 |
| 65 | 4 | 20 | 0.25 | 0.74 | 0.01 |
| 66 | 4 | 15 | 0.25 | 0.74 | 0.01 |
| 67 | 4 | 10 | 0.25 | 0.74 | 0.01 |
| 68 | 4 | 5 | 0.25 | 0.74 | 0.01 |
| 69 | 4 | 20 | 0.20 | 0.74 | 0.01 |
| 70 | 4 | 15 | 0.20 | 0.74 | 0.01 |
| 71 | 4 | 10 | 0.20 | 0.74 | 0.01 |
| 72 | 4 | 5 | 0.20 | 0.74 | 0.01 |
| 73 | 4 | 20 | 0.15 | 0.74 | 0.01 |
| 74 | 4 | 15 | 0.15 | 0.74 | 0.01 |
| 75 | 4 | 10 | 0.15 | 0.74 | 0.01 |
| 76 | 4 | 5 | 0.15 | 0.74 | 0.01 |
| 77 | 4 | 20 | 0.10 | 0.74 | 0.01 |
| 78 | 4 | 15 | 0.10 | 0.74 | 0.01 |
| 79 | 4 | 10 | 0.10 | 0.74 | 0.01 |
| 80 | 4 | 5 | 0.10 | 0.74 | 0.01 |
| 81 | 4 | 20 | 0.25 | 0.74 | 0 |
| 82 | 4 | 15 | 0.25 | 0.74 | 0 |
| 83 | 4 | 10 | 0.25 | 0.74 | 0 |
| 84 | 4 | 5 | 0.25 | 0.74 | 0 |
| 85 | 4 | 20 | 0.20 | 0.74 | 0 |
| 86 | 4 | 15 | 0.20 | 0.74 | 0 |
| 87 | 4 | 10 | 0.20 | 0.74 | 0 |
| 88 | 4 | 5 | 0.20 | 0.74 | 0 |
| 89 | 4 | 20 | 0.15 | 0.74 | 0 |
| 90 | 4 | 15 | 0.15 | 0.74 | 0 |
| 91 | 4 | 10 | 0.15 | 0.74 | 0 |
| 92 | 4 | 5 | 0.15 | 0.74 | 0 |
| 93 | 4 | 20 | 0.10 | 0.74 | 0 |
| 94 | 4 | 15 | 0.10 | 0.74 | 0 |
| 95 | 4 | 10 | 0.10 | 0.74 | 0 |
| 96 | 4 | 5 | 0.10 | 0.74 | 0 |
| 97 | 4 | 20 | 0.25 | 0.64 | 0.01 |
| 98 | 4 | 15 | 0.25 | 0.64 | 0.01 |
| 99 | 4 | 10 | 0.25 | 0.64 | 0.01 |
| 100 | 4 | 5 | 0.25 | 0.64 | 0.01 |
| 101 | 4 | 20 | 0.20 | 0.64 | 0.01 |
| 102 | 4 | 15 | 0.20 | 0.64 | 0.01 |
| 103 | 4 | 10 | 0.20 | 0.64 | 0.01 |
| 104 | 4 | 5 | 0.20 | 0.64 | 0.01 |
| 105 | 4 | 20 | 0.15 | 0.64 | 0.01 |
| 106 | 4 | 15 | 0.15 | 0.64 | 0.01 |
| 107 | 4 | 10 | 0.15 | 0.64 | 0.01 |
| 108 | 4 | 5 | 0.15 | 0.64 | 0.01 |
| 109 | 4 | 20 | 0.10 | 0.64 | 0.01 |
| 110 | 4 | 15 | 0.10 | 0.64 | 0.01 |
| 111 | 4 | 10 | 0.10 | 0.64 | 0.01 |
| 112 | 4 | 5 | 0.10 | 0.64 | 0.01 |
| 113 | 4 | 20 | 0.25 | 0.64 | 0 |
| 114 | 4 | 15 | 0.25 | 0.64 | 0 |
| 115 | 4 | 10 | 0.25 | 0.64 | 0 |
| 116 | 4 | 5 | 0.25 | 0.64 | 0 |
| 117 | 4 | 20 | 0.20 | 0.64 | 0 |
| 118 | 4 | 15 | 0.20 | 0.64 | 0 |
| 119 | 4 | 10 | 0.20 | 0.64 | 0 |
| 120 | 4 | 5 | 0.20 | 0.64 | 0 |
| 121 | 4 | 20 | 0.15 | 0.64 | 0 |
| 122 | 4 | 15 | 0.15 | 0.64 | 0 |
| 123 | 4 | 10 | 0.15 | 0.64 | 0 |
| 124 | 4 | 5 | 0.15 | 0.64 | 0 |
| 125 | 4 | 20 | 0.10 | 0.64 | 0 |
| 126 | 4 | 15 | 0.10 | 0.64 | 0 |
| 127 | 4 | 10 | 0.10 | 0.64 | 0 |
| 128 | 4 | 5 | 0.10 | 0.64 | 0 |
| 129 | 6 | 20 | 0.25 | 0.74 | 0.01 |
| 130 | 6 | 15 | 0.25 | 0.74 | 0.01 |
| 131 | 6 | 10 | 0.25 | 0.74 | 0.01 |
| 132 | 6 | 5 | 0.25 | 0.74 | 0.01 |
| 133 | 6 | 20 | 0.20 | 0.74 | 0.01 |
| 134 | 6 | 15 | 0.20 | 0.74 | 0.01 |
| 135 | 6 | 10 | 0.20 | 0.74 | 0.01 |
| 136 | 6 | 5 | 0.20 | 0.74 | 0.01 |
| 137 | 6 | 20 | 0.15 | 0.74 | 0.01 |
| 138 | 6 | 15 | 0.15 | 0.74 | 0.01 |
| 139 | 6 | 10 | 0.15 | 0.74 | 0.01 |
| 140 | 6 | 5 | 0.15 | 0.74 | 0.01 |
| 141 | 6 | 20 | 0.10 | 0.74 | 0.01 |
| 142 | 6 | 15 | 0.10 | 0.74 | 0.01 |
| 143 | 6 | 10 | 0.10 | 0.74 | 0.01 |
| 144 | 6 | 5 | 0.10 | 0.74 | 0.01 |
| 145 | 6 | 20 | 0.25 | 0.74 | 0 |
| 146 | 6 | 15 | 0.25 | 0.74 | 0 |
| 147 | 6 | 10 | 0.25 | 0.74 | 0 |
| 148 | 6 | 5 | 0.25 | 0.74 | 0 |
| 149 | 6 | 20 | 0.20 | 0.74 | 0 |
| 150 | 6 | 15 | 0.20 | 0.74 | 0 |
| 151 | 6 | 10 | 0.20 | 0.74 | 0 |
| 152 | 6 | 5 | 0.20 | 0.74 | 0 |
| 153 | 6 | 20 | 0.15 | 0.74 | 0 |
| 154 | 6 | 15 | 0.15 | 0.74 | 0 |
| 155 | 6 | 10 | 0.15 | 0.74 | 0 |
| 156 | 6 | 5 | 0.15 | 0.74 | 0 |
| 157 | 6 | 20 | 0.10 | 0.74 | 0 |
| 158 | 6 | 15 | 0.10 | 0.74 | 0 |
| 159 | 6 | 10 | 0.10 | 0.74 | 0 |
| 160 | 6 | 5 | 0.10 | 0.74 | 0 |
| 161 | 6 | 20 | 0.25 | 0.64 | 0.01 |
| 162 | 6 | 15 | 0.25 | 0.64 | 0.01 |
| 163 | 6 | 10 | 0.25 | 0.64 | 0.01 |
| 164 | 6 | 5 | 0.25 | 0.64 | 0.01 |
| 165 | 6 | 20 | 0.20 | 0.64 | 0.01 |
| 166 | 6 | 15 | 0.20 | 0.64 | 0.01 |
| 167 | 6 | 10 | 0.20 | 0.64 | 0.01 |
| 168 | 6 | 5 | 0.20 | 0.64 | 0.01 |
| 169 | 6 | 20 | 0.15 | 0.64 | 0.01 |
| 170 | 6 | 15 | 0.15 | 0.64 | 0.01 |
| 171 | 6 | 10 | 0.15 | 0.64 | 0.01 |
| 172 | 6 | 5 | 0.15 | 0.64 | 0.01 |
| 173 | 6 | 20 | 0.10 | 0.64 | 0.01 |
| 174 | 6 | 15 | 0.10 | 0.64 | 0.01 |
| 175 | 6 | 10 | 0.10 | 0.64 | 0.01 |
| 176 | 6 | 5 | 0.10 | 0.64 | 0.01 |
| 177 | 6 | 20 | 0.25 | 0.64 | 0 |
| 178 | 6 | 15 | 0.25 | 0.64 | 0 |
| 179 | 6 | 10 | 0.25 | 0.64 | 0 |
| 180 | 6 | 5 | 0.25 | 0.64 | 0 |
| 181 | 6 | 20 | 0.20 | 0.64 | 0 |
| 182 | 6 | 15 | 0.20 | 0.64 | 0 |
| 183 | 6 | 10 | 0.20 | 0.64 | 0 |
| 184 | 6 | 5 | 0.20 | 0.64 | 0 |
| 185 | 6 | 20 | 0.15 | 0.64 | 0 |
| 186 | 6 | 15 | 0.15 | 0.64 | 0 |
| 187 | 6 | 10 | 0.15 | 0.64 | 0 |
| 188 | 6 | 5 | 0.15 | 0.64 | 0 |
| 189 | 6 | 20 | 0.10 | 0.64 | 0 |
| 190 | 6 | 15 | 0.10 | 0.64 | 0 |
| 191 | 6 | 10 | 0.10 | 0.64 | 0 |
| 192 | 6 | 5 | 0.10 | 0.64 | 0 |
| 193 | 8 | 20 | 0.25 | 0.74 | 0.01 |
| 194 | 8 | 15 | 0.25 | 0.74 | 0.01 |
| 195 | 8 | 10 | 0.25 | 0.74 | 0.01 |
| 196 | 8 | 5 | 0.25 | 0.74 | 0.01 |
| 197 | 8 | 20 | 0.20 | 0.74 | 0.01 |
| 198 | 8 | 15 | 0.20 | 0.74 | 0.01 |
| 199 | 8 | 10 | 0.20 | 0.74 | 0.01 |
| 200 | 8 | 5 | 0.20 | 0.74 | 0.01 |
| 201 | 8 | 20 | 0.15 | 0.74 | 0.01 |
| 202 | 8 | 15 | 0.15 | 0.74 | 0.01 |
| 203 | 8 | 10 | 0.15 | 0.74 | 0.01 |
| 204 | 8 | 5 | 0.15 | 0.74 | 0.01 |
| 205 | 8 | 20 | 0.10 | 0.74 | 0.01 |
| 206 | 8 | 15 | 0.10 | 0.74 | 0.01 |
| 207 | 8 | 10 | 0.10 | 0.74 | 0.01 |
| 208 | 8 | 5 | 0.10 | 0.74 | 0.01 |
| 209 | 8 | 20 | 0.25 | 0.74 | 0 |
| 210 | 8 | 15 | 0.25 | 0.74 | 0 |
| 211 | 8 | 10 | 0.25 | 0.74 | 0 |
| 212 | 8 | 5 | 0.25 | 0.74 | 0 |
| 213 | 8 | 20 | 0.20 | 0.74 | 0 |
| 214 | 8 | 15 | 0.20 | 0.74 | 0 |
| 215 | 8 | 10 | 0.20 | 0.74 | 0 |
| 216 | 8 | 5 | 0.20 | 0.74 | 0 |
| 217 | 8 | 20 | 0.15 | 0.74 | 0 |
| 218 | 8 | 15 | 0.15 | 0.74 | 0 |
| 219 | 8 | 10 | 0.15 | 0.74 | 0 |
| 220 | 8 | 5 | 0.15 | 0.74 | 0 |
| 221 | 8 | 20 | 0.10 | 0.74 | 0 |
| 222 | 8 | 15 | 0.10 | 0.74 | 0 |
| 223 | 8 | 10 | 0.10 | 0.74 | 0 |
| 224 | 8 | 5 | 0.10 | 0.74 | 0 |
| 225 | 8 | 20 | 0.25 | 0.64 | 0.01 |
| 226 | 8 | 15 | 0.25 | 0.64 | 0.01 |
| 227 | 8 | 10 | 0.25 | 0.64 | 0.01 |
| 228 | 8 | 5 | 0.25 | 0.64 | 0.01 |
| 229 | 8 | 20 | 0.20 | 0.64 | 0.01 |
| 230 | 8 | 15 | 0.20 | 0.64 | 0.01 |
| 231 | 8 | 10 | 0.20 | 0.64 | 0.01 |
| 232 | 8 | 5 | 0.20 | 0.64 | 0.01 |
| 233 | 8 | 20 | 0.15 | 0.64 | 0.01 |
| 234 | 8 | 15 | 0.15 | 0.64 | 0.01 |
| 235 | 8 | 10 | 0.15 | 0.64 | 0.01 |
| 236 | 8 | 5 | 0.15 | 0.64 | 0.01 |
| 237 | 8 | 20 | 0.10 | 0.64 | 0.01 |
| 238 | 8 | 15 | 0.10 | 0.64 | 0.01 |
| 239 | 8 | 10 | 0.10 | 0.64 | 0.01 |
| 240 | 8 | 5 | 0.10 | 0.64 | 0.01 |
| 241 | 8 | 20 | 0.25 | 0.64 | 0 |
| 242 | 8 | 15 | 0.25 | 0.64 | 0 |
| 243 | 8 | 10 | 0.25 | 0.64 | 0 |
| 244 | 8 | 5 | 0.25 | 0.64 | 0 |
| 245 | 8 | 20 | 0.20 | 0.64 | 0 |
| 246 | 8 | 15 | 0.20 | 0.64 | 0 |
| 247 | 8 | 10 | 0.20 | 0.64 | 0 |
| 248 | 8 | 5 | 0.20 | 0.64 | 0 |
| 249 | 8 | 20 | 0.15 | 0.64 | 0 |
| 250 | 8 | 15 | 0.15 | 0.64 | 0 |
| 251 | 8 | 10 | 0.15 | 0.64 | 0 |
| 252 | 8 | 5 | 0.15 | 0.64 | 0 |
| 253 | 8 | 20 | 0.10 | 0.64 | 0 |
| 254 | 8 | 15 | 0.10 | 0.64 | 0 |
| 255 | 8 | 15 | 0.10 | 0.64 | 0 |
| 256 | 8 | 5 | 0.10 | 0.64 | 0 |

*Nlxn. = naloxone HCl

### C. Devices

Also provided herein are spray devices containing the solutions described herein. In certain embodiments, these devices are pre-primed, single use devices. More than one device may be packaged together enabling the delivery of more than one dose if it is needed. Non-limiting examples of devices suitable for use in delivering the solutions described above can be found in U.S. Pat. No. 5,307,953 to Regan, and U.S. Pat. No. 4,946,069 to Fuchs, each of which is incorporated by reference in its entirety.

In certain embodiments, the device comprises a reservoir with a volume between about 80 μL and about 450 μL, for example about 90 μL, about 100 μL, about 110 μL, about 120 μL, about 130 μL, about 140 μL, about 150 μL, about 200 μL, about 250 μL, about 300 μL, about 350 μL, or about 400 μL. The volume of the solution in the reservoir cannot exceed the reservoir volume, but the volume of solution can be less than the reservoir volume. For example, in certain embodiments the volume of solution will be between about 100 μL and about 140 μL, for example between about 110 μL and about 130 μL, for example about 110 μL, about 115 μL, about 120 μL, about 125 μL, or about 130 μL. In certain embodiments, the device will not be able to deliver all solution in the reservoir, and therefore if a given amount of solution must be delivered into the nostril, then a certain amount extra should be stored in the device reservoir. For example, in certain embodiments, to deliver about 100 μL per spray into the nostril it will be necessary to store about 125 μL in the device. In certain embodiments, the device comprises a plunger that houses a container closure with a vial having an opening, a cannula, and a rubber stopper. The stopper can be configured to occlude the opening of the vial, and the cannula can be configured such that the cannula can pierce the stopper when the plunger applies sufficient force to the cannula.

In certain embodiments, the device can be a single-use device. More than one device may be packaged together enabling the delivery of more than one dose if it is needed. One advantage of single-use devices is that they deliver only a specified amount of solution, so that the user does not need to make adjustments prior to drug delivery. In certain embodiments, the device can be a pre-primed device. One advantage of pre-primed devices is that they are immediately ready for use, and therefore can save valuable seconds when administering naloxone to a patient is suffering from respiratory depression.

In certain embodiments, the device is configured to deliver two doses of solution, for example an APTAR® BDS® bi-dose device. In certain embodiments, the device is configured to deliver multiple doses, for example 3 doses, 4 doses, 5 doses, 6 doses, 7 doses, 8 doses, 9 doses, or 10 or more doses. In certain bi-dose embodiments, the device can be configured to deliver two doses simultaneously (e.g., one to each nostril), or two doses in series, one after the other (e.g., the user administers one dose to one nostril, then moves the device to the second nostril and administers a second dose, or administers two doses in series in the same nostril). In certain embodiments, the volume of solution administered in each dose is identical (e.g., both about 50 μL, both about 100 μL, both about 150 μL, or both about 200 μL), while in other embodiments the volumes of the two doses are different (e.g., first dose about 100 μL and second about 50 μL, or first about 200 μL and second about 100 μL). More than one bi-dose (or multiple dose) devices may be packaged together enabling multiple doses to be administered to the patient if required.

In certain embodiments, devices carrying different concentrations of solution can be manufactured in different colors. For example, devices containing solution to deliver 4 mg dose of naloxone can be partially or totally pink (for example, a pink plunger with a white grip and white nozzle), while a device containing solution to deliver a 2 mg dose of naloxone can be all or substantially all white.

### D. Methods of Use

In certain embodiments, the solutions and devices described herein can be used to treat opioid overdose. One advantage of the solutions and devices described herein is that they are acceptable for intranasal administration of naloxone. Intranasal administration of naloxone has certain advantages over injection, in that nasal administration requires less, or no, medical training and hence can be used in community settings. This is in contrast to injections which may require medical training and/or actions to prepare the injection. Hence nasal delivery has substantial advantages, particularly when used in community settings, for example in the high stress situation when a family member or friend needs to administer naloxone to an overdosed individual. Speed and simplicity of administration clearly also matters when looking to reverse an overdose in order to prevent organ damage or death which can arise with respiratory depression. In addition, nasal administration minimizes the likelihood of accidental needle sticks and the corresponding danger of blood-borne infection. Intranasal administration also has certain advantages over buccal or sublingual administration, in that intranasal administration does not require the caregiver to insert fingers into a patient's mouth, thus minimizing the likelihood of bite injuries to the caregiver.

Using the devices described herein, the caregiver can disperse a mist of solution described above into the nasal cavity of a patient exhibiting symptoms of opioid overdose. One of the most prominent symptoms of opioid overdose is respiratory depression. Significantly, because the solutions described herein can be absorbed through the membranes of the nasal cavity, the solutions and devices described herein do not require that the patient be breathing to inhale the naloxone in order for the naloxone to have effect. Where the device described herein is a pre-primed device such as an APTAR device or a BECTON-DICKINSON device, the caregiver need only insert the spray nozzle into the patient's nostril and depress the device button to disperse a full dose in a spray of appropriate shape and particle size. Because the average adult nasal cavity has a volume of approximately 250 μL, it is important that the solution delivered be concentrated enough to fit into a volume less than about 250 μL (e.g., about 200 μL, about 150 μL, about 100 μL, or about 50 μL).

Once the caregiver has administered a single dose of naloxone using a device and/or solution described herein, the caregiver can check the patient to determine whether the naloxone has reversed the overdose. Where the patient still exhibits overdose symptoms, the caregiver can administer a second dose, and subsequent doses as necessary, until the patient shows signs of the overdose reversing. In certain embodiments, the caregiver will alternate nostrils while administers second and subsequent doses.

In certain embodiments, the devices and solutions described herein can be used to reverse overdoses from fentanyl or a fentanyl derivative. Fentanyl derivatives can be very potent. Some fentanyl derivatives, such as carfentanyl, are about 100 times more powerful than fentanyl and 10,000 times more powerful that heroin (diamorphine). A caregiver will need, therefore, to administer correspondingly more naloxone to reverse an overdose induced by these highly potent fentanyl derivatives. In certain embodiments, the caregiver will administer more doses of a lower concentration (e.g., 2 mg/mL) of naloxone solution, while in other embodiments the caregiver will administer fewer doses of a higher concentration (e.g., 6 mg/mL) solution.

In certain embodiments, the solutions and devices described herein can be used to prevent opioid users from titrating opioid receptor occupancy. In certain embodiments, the devices disclosed herein deliver only a certain, fixed, prespecified dose. For example, where the device used in an APTAR® UnitDose® or BiDose® device, about 100 μL of solution will be delivered per spray, such that the caregiver has no means to deliver more or less than the fixed dose enabled by such device. In this way, the caregiver is precluded from trying to administer just enough naloxone to reverse overdose. By distributing nasal-spray naloxone in these fixed dose devices—instead of in injectable form—public health authorities can prevent caregivers from reversing overdose insufficiently.

In certain embodiments, the solutions and devices described herein can be used under low-temperature conditions. In certain embodiments, the solutions and devices are used after an extended period (e.g., at least about 10 hrs., at least about 24 hrs., at least about 48 hrs., at least about 1 week, at least about 2 weeks, at least about 1 month, or at least about 5 months) in storage at low temperature (e.g., less than about 4° C., less than about 0° C., less than about −5° C., less than about −10° C., less than about −15° C., or less than about −20° C.). In certain embodiments, the solution freezes during storage at low temperature, but thaws quickly when needed and is sufficiently intact to remain usable.

An advantage of the methods disclosed herein is that they achieve an acceptably high serum concentration of the opioid antagonist very quickly. For example, in certain embodiments the plasma concentration versus time curve of opioid antagonist (e.g., naloxone) in the patient has a tₘₐₓ of less than 30 minutes. In certain embodiments, the patient experiences a geometric mean naloxone Cₘₐₓ not less than about 3 ng/mL following a single spray. In certain embodiments, the patient experiences a plasma naloxone concentration such that the geometric mean of area under a plasma concentration versus time curve (AUC_{0-∞}) is not less than about 8 hr*ng/mL when time is extrapolated to infinity.

### E. Exemplary Embodiments

The present disclosure further provides the following non-limiting embodiments.

### Embodiment 1

An aqueous solution comprising: an opioid antagonist, between about 2% (w/v) and about 25% (w/v) propylene glycol (PG), and between about 0.2% (w/v) and about 1.2% (w/v) of an isotonicity agent, wherein the solution comprises no more than about 2% (w/v) of alcohol (for example no more than about 1% w/v of alcohol), and wherein the solution has a dynamic viscosity less than about 100 cP at 21° C., and optionally wherein the solution contains no more than about 0.09% (w/v) of alkyl paraben, for example no more than about 0.06% (w/v), no more than about 0.02% (w/v), or no more than about 0.005% (w/v).

### Embodiment 2

The solution of Embodiment 1, wherein the opioid antagonist is selected from the group consisting of naloxone, naltrexone, nalmefene, diprenorphine, nalorphine, nalorphine dinicotinate, levallorphan, samidorphan, nalodeine, and a combinations of any one or more of the above listed opioid antagonists, as well as pharmaceutically acceptable salts and/or solvates thereof.

### Embodiment 3

The solution of Embodiment 1, wherein the opioid antagonist is naloxone, or a pharmaceutically acceptable salt and/or solvate thereof, such as naloxone HCl.

### Embodiment 4

An aqueous solution comprising: between about 2% (w/v) and about 12% (w/v) naloxone or a pharmaceutically acceptable salt thereof, between about 2% (w/v) and about 25% (w/v) propylene glycol (PG), and isotonicity agent sufficient to achieve an osmolality between about 300 mOsm and 2500 mOsm; wherein the solution comprises no more than about 2% (w/v) of alcohol, wherein the solution has a dynamic viscosity less than about 100 cP at 21° C., and wherein the solution does not contain butyl paraben, methyl paraben, ethyl paraben, propyl paraben, sodium benzoate, or benzoic acid.

### Embodiment 5

The solution of any one of Embodiments 1-4, further comprising between about 0.05% and about 1% (w/v) of a stabilizing agent, optionally between about 0.05% and about 0.2% (w/v).

### Embodiment 6

The solution of any one of Embodiments 1-4, wherein the solution contains no stabilizing agent.

### Embodiment 7

The solution of any one of Embodiments 1-6, wherein the solution comprises at least about 4% (w/v), for example at least about 5% (w/v), at least about 6% (w/v), at least about 7% (w/v), at least about 8% (w/v), at least about 9% (w/v), at least about 10% (w/v), at least about 11% (w/v) or about 12% (w/v) naloxone or a pharmaceutically acceptable salt thereof.

### Embodiment 8

The solution of any one of Embodiments 1-7, wherein the solution comprises at least about 4% (w/v) naloxone or a pharmaceutically acceptable salt thereof.

### Embodiment 9

The solution of any one of Embodiments 1-8, wherein the PG is present in a concentration between about 5% and about 10% (w/v).

### Embodiment 10

The solution of any one of Embodiments 1-9, wherein the PG is present in a concentration between about 15% and about 20% (w/v).

### Embodiment 11

The solution of any one of Embodiments 1-10, wherein osmolality is between about 350 mOsm and about 2500 mOsm.

### Embodiment 12

The solution of any one of Embodiments 1-11, wherein osmolality is between about 1 Osm and about 2.5 Osm.

### Embodiment 13

The solution of any one of Embodiments 1-12, wherein the stabilizing agent is present in a concentration between about 0.05% and about 0.15% (w/v).

### Embodiment 14

The solution of any one of Embodiments 1-13, wherein the isotonicity agent is present in a concentration between about 0.6% and about 1% (w/v).

### Embodiment 15

The solution of any one of Embodiments 1-14, further comprising an amount of acid or buffer sufficient to achieve a pH between about 3 and about 7.

### Embodiment 16

The solution of any one of Embodiments 1-15, wherein the solution further comprises sorbitol, for example about 3% (w/v), about 4% (w/v), about 4.5% (w/v), or about 5% (w/v) sorbitol.

### Embodiment 17

The solution of any one of Embodiments 1-16, wherein: the isotonicity agent is sodium chloride; the stabilizing agent is disodium edetate; and the acid is hydrochloric acid.

### Embodiment 18

The solution of any one of Embodiments 1-17, wherein the solution comprises no detectable amount of one or more selected from the group consisting of alcohol, butyl paraben, methyl paraben, ethyl paraben, propyl paraben, sodium benzoate, and benzoic acid.

### Embodiment 19

The solution of any one of Embodiments 1-18, wherein the solution comprises no detectable emulsifier.

### Embodiment 20

The solution of any one of Embodiments 1-19, further comprising between about 0.005% and about 0.015% (w/v) of a preservative.

### Embodiment 21

The solution of any one of Embodiments 1-20, wherein the preservative is about 0.01% (w/v) benzalkonium chloride, and wherein the pH is between about 3.5 and about 5.5.

### Embodiment 22

The solution of any one of Embodiments 1-21, wherein the solution does not contain any additional preservative, beyond the ingredients claimed, and wherein the pH is between about 3.5 and about 5.5.

### Embodiment 23

The solution of any one of Embodiments 1-22, wherein the solution comprises: about 4% (w/v) naloxone HCl; between about 0.6% (w/v) and about 0.8% (w/v) NaCl; about 5% (w/v) PG; and between about 0.05% (w/v) and about 0.2% (w/v) disodium edetate.

### Embodiment 24

The solution of any one of Embodiments 1-23, wherein the solution comprises: about 4% (w/v) naloxone HCl; between about 0.6% (w/v) and about 0.8% (w/v) NaCl; about 10% (w/v) PG; and between about 0.05% (w/v) and about 0.2% (w/v) disodium edetate.

### Embodiment 25

The solution of any one of Embodiments 1-24, wherein the solution comprises: about 4% (w/v) naloxone HCl; between about 0.6% (w/v) and about 0.8% (w/v) NaCl; about 5% (w/v) PG; and between about 0.05% (w/v) and about 0.2% (w/v) disodium edetate.

### Embodiment 26

The solution of any one of Embodiments 1-25, wherein the solution comprises: about 4% (w/v) naloxone HCl; between about 0.6% (w/v) and about 0.8% (w/v) NaCl; about 10% (w/v) PG; and between about 0.05% (w/v) and about 0.2% (w/v) disodium edetate.

### Embodiment 27

The solution of any one of Embodiments 1-26, wherein the solution comprises: about 4% (w/v) naloxone HCl; between about 0.6% (w/v) and about 0.8% (w/v) NaCl; about 15% (w/v) PG; and between about 0.05% (w/v) and about 0.2% (w/v) disodium edetate.

### Embodiment 28

The solution of any one of Embodiments 1-27, wherein the solution comprises: about 4% (w/v) naloxone HCl; between about 0.6% (w/v) and about 0.8% (w/v) NaCl; about 20% (w/v) PG; and between about 0.05% (w/v) and about 0.2% (w/v) disodium edetate.

### Embodiment 29

The solution of any one of Embodiments 1-28, wherein the solution consists essentially of: about 4% (w/v) naloxone HCl; between about 0.6% (w/v) and about 0.8% (w/v) NaCl; about 5% (w/v) PG; and between about 0.05% (w/v) and about 0.2% (w/v) disodium edetate.

### Embodiment 30

The solution of any one of Embodiments 1-29, wherein the solution consists essentially of: about 4% (w/v) naloxone HCl; between about 0.6% (w/v) and about 0.8% (w/v) NaCl; about 10% (w/v) PG; and between about 0.05% (w/v) and about 0.2% (w/v) disodium edetate.

### Embodiment 31

The solution of any one of Embodiments 1-30, wherein the solution consists essentially of: about 4% (w/v) naloxone HCl; between about 0.6% (w/v) and about 0.8% (w/v) NaCl; about 5% (w/v) PG; and between about 0.05% (w/v) and about 0.2% (w/v) disodium edetate.

### Embodiment 32

The solution of any one of Embodiments 1-31, wherein the solution consists essentially of: about 4% (w/v) naloxone HCl; between about 0.6% (w/v) and about 0.8% (w/v) NaCl; about 10% (w/v) PG; and between about 0.05% (w/v) and about 0.2% (w/v) disodium edetate.

### Embodiment 33

The solution of any one of Embodiments 1-32, wherein the solution has a volume of about 80 μL to about 150 μL, and wherein the solution comprises: about 4% (w/v) naloxone HCl; about 0.5% (w/v) to about 1% (w/v) NaCl; about 5% (w/v) to about 10% (w/v) PG; and hydrochloric acid sufficient to achieve a pH of 3.5-5.5.

### Embodiment 34

The solution of any one of Embodiments 1-22, wherein the solution comprises at least about 6% (w/v) naloxone or a pharmaceutically acceptable salt thereof.

### Embodiment 35

The solution of any one of Embodiments 1-22, wherein the solution comprises at least about 8% (w/v) naloxone or a pharmaceutically acceptable salt thereof.

### Embodiment 36

The solution of Embodiment 35, wherein the solution further comprises between 0.05% (w/v) and 0.2% (w/v) of EDTA.

### Embodiment 37

The solution of Embodiment 36, wherein the solution does not comprise EDTA.

### Embodiment 38

The solution of anyone of any one of the previous embodiments, wherein the solution comprises no more than about 1.0% impurities (for example, no more than about 0.5%, no more than about 0.1%, or no more than about 0.05%), wherein the concentration of impurities is based on total area percentage measured by reversed phase-high pressure liquid chromatography (RP-HPLC).

### Embodiment 39

The solution of Embodiment 38, wherein the solution comprises at least one impurity with a relative retention time of less than 1, or less than 0.75, or less than 0.5, when the solution is evaluated by high pressure liquid chromatography, or wherein the solution comprises at least one impurity with a relative retention time greater than 1, or greater than 1.5, or greater than 1.8, when the solution is evaluated by high pressure liquid chromatography.

### Embodiment 40

A pre-primed, single-use nasal spray device, wherein the device comprises a reservoir, a piston, a swirl chamber, and a spray nozzle; and wherein the reservoir contains the solution of any one of Embodiments 1-39.

### Embodiment 41

The device of Embodiment 40, wherein the device has a reservoir containing approximately 125 μL of the solution.

### Embodiment 42

A pre-primed, two-use nasal spray device, wherein the device comprises a reservoir, a piston, a swirl chamber, and a spray nozzle; and wherein the reservoir contains the solution of any one of Embodiments 1-39.

### Embodiment 43

The device of Embodiment 42, wherein the device has a reservoir containing approximately 225 μL of the solution.

### Embodiment 44

A pre-primed, multi-use nasal spray device, wherein the device comprises a reservoir, a piston, a swirl chamber, and a spray nozzle; and wherein the reservoir contains the solution of any one of Embodiments 1-39.

### Embodiment 45

A mist, wherein the mist stands adjacent to a spray nozzle, wherein the mist comprises droplets of the solution of any one of Embodiments 1-39, wherein no more than about 10% of the droplets have a diameter less than 10 m as measured by laser diffraction at 3 cm and 6 cm from the spray nozzle.

### Embodiment 46

The mist of Embodiment 45, wherein the mist takes the shape of a round plume with an ovality ratio less than 2.0.

### Embodiment 47

The mist of Embodiment 45 or 46, wherein the naloxone is at least 40% bioavailable.

### Embodiment 48

The mist of any one of Embodiments 45-47, wherein the median droplet size is between about 30 μm and about 100 μm.

### Embodiment 49

The mist of any one of Embodiments 45-48, wherein approximately 50% of droplets have a diameter between about 30 m and about 70 m.

### Embodiment 50

The mist of any one of Embodiments 45-49, wherein approximately 90% of droplets have a diameter less than about 100 m.

### Embodiment 51

The mist of any one of Embodiments 45-50, wherein no more than approximately 2% of droplets have a diameter less than about 10 m.

### Embodiment 52

A method of treating opioid overdose in a patient in need thereof, the method comprising: delivering a spray from a pre-primed, single-use nasal spray device into a nostril of the patient, wherein a reservoir of the device contains the solution of any one of Embodiments 1-39.

### Embodiment 53

The method of Embodiment 52, wherein the plasma concentration versus time curve of naloxone in the patient has a tₘₐₓ of less than 30 minutes.

### Embodiment 54

The method of Embodiment 52 or 53, wherein In certain embodiments, the patient experiences a geometric mean naloxone Cₘₐₓ not less than about 3 ng/mL following a single spray.

### Embodiment 55

The method of any one of Embodiments 52-54, wherein the patient experiences a plasma naloxone concentration such that the geometric mean of area under a plasma concentration versus time curve (AUC_{0-∞}) is not less than about 8 hr*ng/mL when time is extrapolated to infinity.

### Embodiment 56

The method of any one of Embodiments 52-55, wherein the opioid is fentanyl or a fentanyl derivative.

### Embodiment 57

The method of any one of Embodiments 52-56, wherein the solution is stored at a temperature of about 0° C. or less for at least one hour, for example at least one day, at least one week, or at least one month, prior to administration.

### Embodiment 58

The method of Embodiment 57 wherein the solution is stored at a temperature of about −5° C. or less for at least one hour, for example at least one day, at least one week, or at least one month, prior to administration.

### Embodiment 59

A method of preventing the use of naloxone to titrate opioid receptor occupancy, the method comprising: actuating the pre-primed, single use device of any one of Embodiments 40-44 to deliver a spray into a nostril of a patient.

### EXAMPLES

### Example 1: Preparation of Formulations

Without further description, it is believed that one of ordinary skill in the art can, using the preceding description and the following illustrative examples, make and utilize the solutions and devices described herein and practice the methods disclosed herein.

Exemplary nasal spray formulations for use in the following experiments were prepared as follows: all excipients (see, Table 3 below) are dissolved in water to achieve a volume approximately 10% less than the target volume. The pH is then adjusted to between about 3 and about 7. The solution is sonicated for about 10 minutes to ensure complete dissolution of solid materials. Finally, the remainder of the water is added to reach target volume and its pH is verified for a second time.

TABLE 3
Nasal spray formulations in 1 mL

| | | Naloxone HCl strength | |
|---|---|---|---|
| | | 20 mg/mL | 40 mg/mL |
| Component | Grade | Quantity/mL | Quantity/mL |
| Naloxone HCl•2H₂O | USP | 22 mg | 44 mg |
| (as Naloxone HCl) | | 20 mg | 40 mg |
| BZK | USP | 0.1 mg | 0.1 mg |
| Na₂EDTA | USP | 2.0 mg | 2.0 mg |
| NaCl | USP | 7.4 mg | 7.4 mg |
| 1N HCl | USP | Adjust to pH 4.5 | Adjust to pH 4.5 |
| Purified H₂O | USP | q.s. to 1 mL | q.s. to 1 mL |

Separate media formulations containing PG (20% w/v) were prepared and adjusted to pH 4.5 with 1N HCl. Naloxone 40 mg/mL (or 44 mg/mL of naloxone HCl dihydrate) composition was chosen for subsequent trials.

### Example 2: Freezing Point Depression Evaluation

Trial formulations were tested in triplicate and a blank (containing no naloxone) was run in parallel. The experiment was performed on a Crystal 16™, where 1 mL of each solution was pipetted into a HPLC vial. The temperature progressively lowered. The cooling program was set as follows: (a) solutions cooled to 5° C. (hold for 1 hour), (b) further cooled to 0° C. (hold for 1 hour), (c) further cooled to −5° C. (hold for 1 hour), (d) further cooled to −10° C. (hold for 1 hour), (e) further cooled to −15° C. (hold for 1 hour), and (f) further cooled to −20° C. This trial was run both with and without magnetic stirring (700 rpm). The transmission of light was measured through each HPLC vial. A transmission value of 100% indicated that the solution was clear and transparent. A transmission value of 0% indicated that the laser light could no longer effectively pass through the HPLC vial because of some freezing or precipitation event. The Crystal 16™ recorded the temperature at which 0% transmission was achieved and these values for each solution tested are documented in Table 4 below.

TABLE 4
Temperature at which solution reaches 0% light transmission

| | | | Transmission = 0% | |
|---|---|---|---|---|
| Naloxone | | | Agitation | No |
| HCl | Formulation | Sample | (700 rpm) | agitation |
| 40 mg/mL | Table 3 | No naloxone | −4.9° C. | ND |
| | formulation | HCl (blank) | | |
| | | Naloxone HCl | −4.0° C. | −10.0° C. |
| | | | −4.9° C. | −8.5° C. |
| | | | −5.7° C. | −9.8° C. |
| | Table 3 | No naloxone | −9.5° C. | ND |
| | formulation + | HCl (blank) | | |
| | PEG₄₀₀ | Naloxone HCl | 5.0° C.* | ND |
| | (20% w/v) | | 5.0° C.* | ND |
| | | | 5.0° C.* | −10.4° C.* |
| | Table 3 | No naloxone | −13.4° C. | ND |
| | formulation + | HCl (blank) | | |
| | PG (20% w/v) | Naloxone HCl | 0° C. | ND |
| | | | −14.9° C. | ND |
| | | | −9.9° C. | ND |

*Transmission occluded by precipitation rather than freezing.

The results from the experiments on the Crystal16™ indicate that the Table 3 formulation freezes at approximately −5° C. (with stirring) and approximately −9.5° C. (without stirring). The addition of 20% (w/v) polyethylene glycol (PEG₄₀₀) lowered the freezing temperature of the blank to −9.5° C., however when naloxone HCl was present the precipitation occurred at temperatures as high as +5.0° C. This called into question the solubility of naloxone HCl in media containing PEG₄₀₀ at low temperatures, and therefore PEG₄₀₀ was abandoned as potential cosolvent. This was further supported by results from samples that were kept at 4° C. for an extended period of time (see below).

By contrast, media containing 20% (w/v) PG revealed more promising results. The formulation containing naloxone HCl reached freezing temperatures of −9.9° C. and −14.9° C. in two of the reaction vials. It is evident that agitation has an influence on the freezing kinetics. Given the promising low temperature freezing results observed for formulation media containing 20% w/v PG, experiments were also performed across a range (10%, 20%, and 30% w/v) of PG concentrations. The results of which are recorded in Table 5.

TABLE 5
Temperature for 0% transmission at 700 RPM agitation

| Naloxone | | | |
|---|---|---|---|
| HCl | Formulation | Sample | Temperature |
| 40 mg/mL | Table 3 | No naloxone | Not reached |
| | formulation + | HCl (blank) | |
| | PG (10% w/v) | Naloxone | Not reached |
| | | | Not reached |
| | | | Not reached |
| | Table 3 | No naloxone | Not reached |
| | formulation + | HCl (blank) | |
| | PG (20% w/v) | Naloxone | Not reached |
| | | | Not reached |
| | | | −4.9* |
| | Table 3 | No naloxone | Not reached |
| | formulation + | HCl (blank) | |
| | PG (30% w/v) | Naloxone | −4.9* |
| | | | −1.5* |
| | | | −2.6* |

*Signifies a precipitation event which affected light transmission, as opposed to freezing.

### Example 3: Freezing Point Suppression Experiments

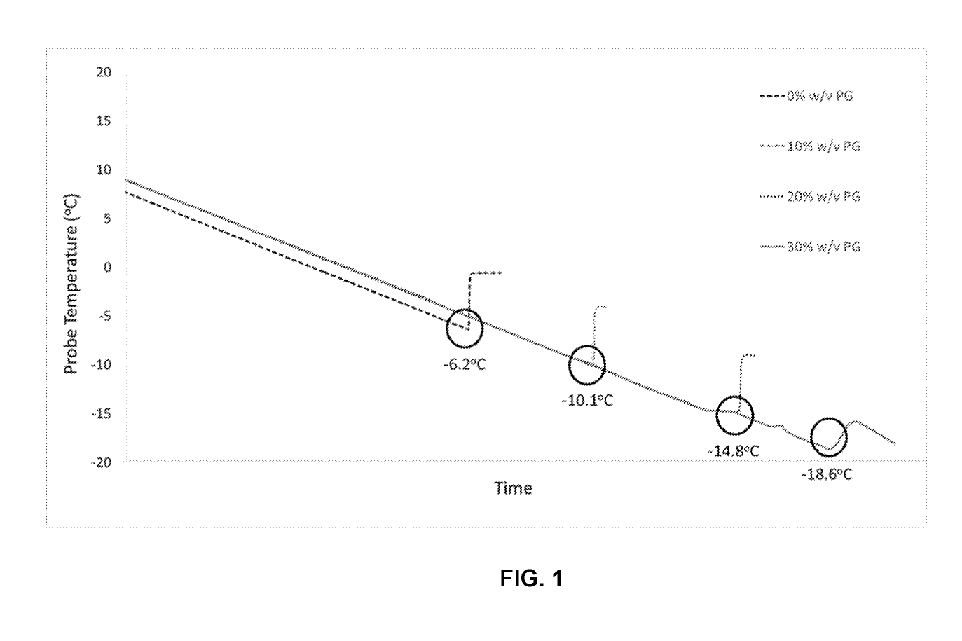

In parallel with the Crystal 16™ experiments, additional freezing point testing was carried out on a Mettler Toledo EasyMax® (see FIG. 1) at different concentrations of PG (0%, 10%, 20%, and 30% w/v). The EasyMax® can reach temperatures as low as −40° C. One can visually monitor the reaction vessel and an in-line temperature probe may be inserted into the reagent tubes. Accordingly, samples (10 mL) with a naloxone HCl concentration of 40 mg/mL were added to reagent tubes. The temperature cooling program was set as follows (unless otherwise stated): solutions were cooled to 10° C. and held for 10 minutes, then solutions were continually cooled at a rate of 0.3 C°/min until the solutions solidified. The temperature trends were monitored during the cool down process. At the point of freezing an approximate+5 C° exothermic event was observed on the EasyMax® software (FIG. 1), thus allowing for easy determination of exact freezing points. Experiments were performed both with agitation (700 RPM) and without agitation. Results are tabulated in Table 6.

TABLE 6
Freezing points evaluation

| PG | Naloxone | Without Stirring | Stirring @ 700 rpm |
|---|---|---|---|
| (w/v) | HCl (mg/mL) | (° C.) | (° C.) |
| 0% | 0 | −9.7 | −6.3 |
| 0% | 40 | −9.0 | −6.2 |
| 10% | | −15.0 | −10.1 |
| 20% | | −16.5 | −14.8 |
| 30% | | −20.2 | −18.6 |

As referred to previously, the FDA IID nasal limit for propylene glycol is 20% w/w. Formulations at this concentration consistently show a freezing point of approximately −15° C. (see, Table 7, both with and without stirring), further confirming the results in Tables 3 and 4 above.

TABLE 7
Freezing points determined on EasyMax ®

| Composition | | | | | | | |
|---|---|---|---|---|---|---|---|
| (per mL) | | | | | Cycle # | | |
| | | | PG | # | 1 | 2 | 3 |
| Naloxone | NaCl | EDTA- | (% | freeze/thaw | Freezing point | | |
| (mg) | (mg) | Na₂ (mg) | w/v) | cycles | (° C.) | | |
| 40 | 7.4 | 1.0 | 20 | 7 | −14.5 | −16 | −15.1 |
| | | 0.0 | 20 | 3 | −16 | −14.2 | −13.3 |
| | | 1.0 | 10 | 3 | −11.3 | −11.9 | −11.3 |
| | | 0.0 | 10 | 3 | −7.9 | −11.7 | −12.2 |
| | | 1.0 | 5 | 3 | −5.9 | −7.1 | −5.9 |
| | | 0.0 | 5 | 3 | −9.1 | −9.7 | −10.2 |

However, the solution remained opaque when thawed, and precipitation was evident. Furthermore, this precipitation event was observed for all solutions containing naloxone HCl in 10%, 20%, and 30% (w/v) PG formulations (see, Table 8). Crucially however, no precipitation occurred in the blank formulations. This indicated that the precipitation was a result of the naloxone HCl being present in the formulation. The precipitate could not be re-dissolved in solution following sonication.

TABLE 8
Naloxone HCl precipitation

| Composition (per mL) | | | | | |
|---|---|---|---|---|---|
| NaCl | EDTA- | PG | EtOH | Freeze/thaw | Naloxone |
| (mg) | Na₂ (mg) | (% w/v) | (% w/v) | cycles (#) | precipitation? |
| 7.4 | 2.0 | 10 | 0 | 1 | yes |
| 7.4 | 2.0 | 20 | 0 | 1 | yes |
| 7.4 | 2.0 | 30 | 0 | 1 | yes |
| 7.4 | 2.0 | 20 | 2 | 1 | yes |
| 7.4 | 2.0 | 20 | 5 | 1 | yes |
| 7.4 | 2.0 | 20 | 10 | 1 | yes |
| 3.7 | 2.0 | 20 | 0 | 1 | yes |
| 7.4 | 1.0 | 20 | 0 | 7 | no |
| 7.4 | 1.0 | 20 | 0 | 4 | no |
| 7.4 | 1.0 | 20 | 0 | 4 | no |
| 7.4 | 0.0 | 20 | 0 | 3 | no |
| 7.4 | 1.0 | 10 | 0 | 3 | no |
| 7.4 | 0.0 | 10 | 0 | 3 | no |
| 7.4 | 1.0 | 5 | 0 | 3 | no |
| 7.4 | 0.0 | 5 | 0 | 3 | No |

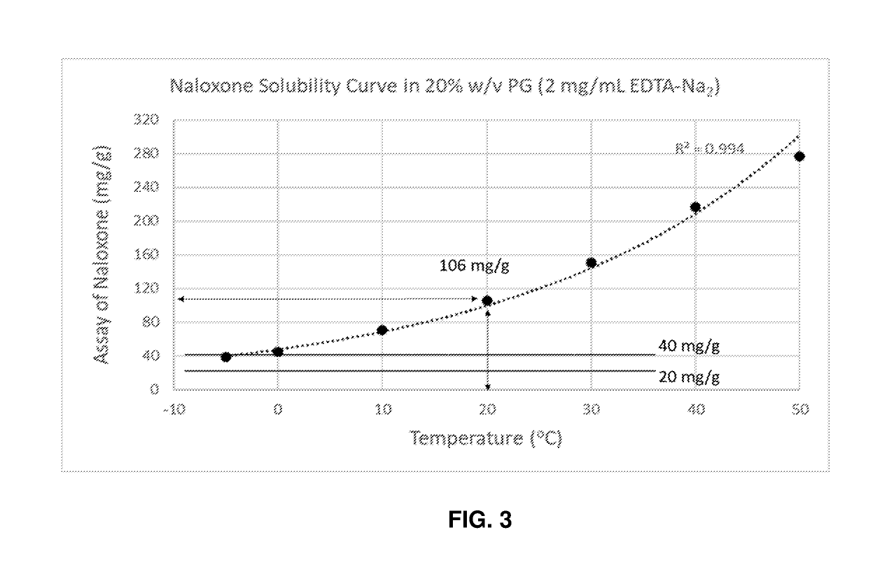

The solubility of naloxone HCl dihydrate in the media containing 20% (w/v) PG is shown in FIG. 3. Saturated solutions of naloxone HCl in the indicated media were held at 7 different temperatures ranging from −5° C. to 50° C., with stirring at 700 RPM overnight. The formulations were filtered through a 0.2 μm polytetrafluoroethylene (PTFE) filter and analyzed by HPLC.

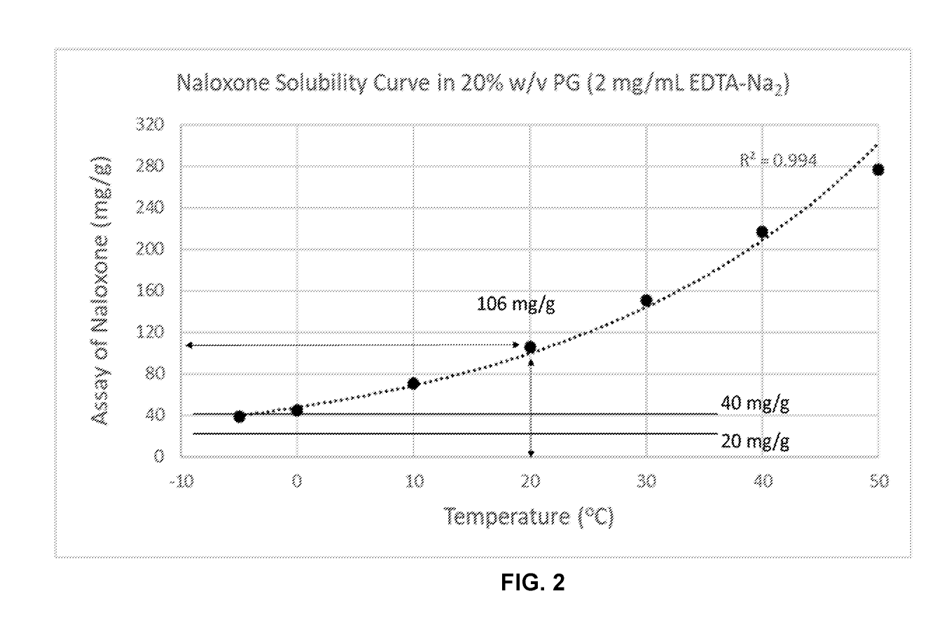

Comparison of the naloxone solubility curves for the original formulation media (FIG. 2) and the formulation containing 20% (w/v) PG (FIG. 3) shows that the effect of adding 20% (w/v) PG to the media lowers the naloxone solubility from approximately 84 mg/mL to 70 mg/mL at 20° C. The 40 mg/mL naloxone HCl formulation containing 20% (w/v) PG reaches saturation at 8° C., as opposed to −15° C. for the media without PG. The 20 mg/mL naloxone HCl formulation containing 20% (w/v) PG approaches saturation at −5° C., as opposed to −25° C. or lower for the media without PG.

Precipitation is sometimes observed when a formulation without PG is stored for approximately 5 days at 4° C. Stability at 4° C. storage can be increased considerably by the addition of PG. Moreover, the freezing temperature of the solution decreases with higher PG concentration. However, higher PG requires a corresponding decrease in EDTA concentration to prevent precipitation (Table 9).

TABLE 9
Naloxone stability during storage at 4° C.

Composition per 100 mL

| | | |
|---|---|---|
| EDTA- | PG | Observed naloxone precipitation |

| Na₂ (mg) | (% w/v) | 24 days | 35 days | 53 days |
|---|---|---|---|---|
| 100 | 20 | No | No | No |
| | | No | Yes | Yes |
| | | Slight | Yes | Yes |
| 0 | 20 | No | No | No |
| | | No | No | No |
| | | No | No | No |
| 100 | 10 | No | No | No |
| | | No | No | No |
| | | No | No | Slight |
| 0 | 10 | No | No | No |
| | | No | No | No |
| | | No | No | No |
| 100 | 5 | No | No | No |
| | | No | No | No |
| | | No | No | No |
| 0 | 5 | No | No | No |
| | | No | No | No |
| | | No | No | No |

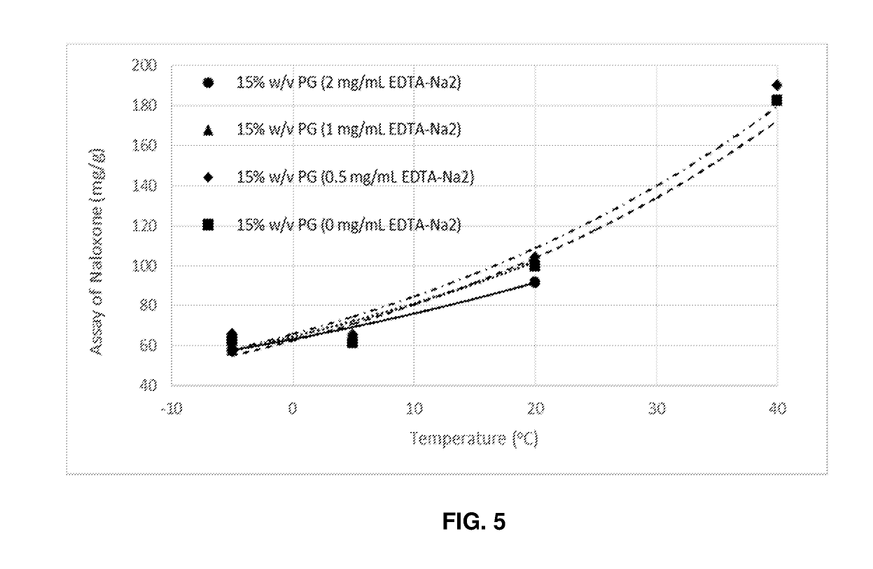

Reducing the level of EDTA does not substantially affect the freezing temperature of the solution (FIG. 5). However, surprisingly, there appears to be an upper limit on the concentration of PG that can be added before provoking naloxone precipitation. The upper limit of tolerable PG concentration can be raised, however, by decreasing EDTA concentration.

### Example 4: Effects of Different EDTA Concentrations

Saturated solutions of naloxone HCl in the 20% (w/v) PG formulation were held at −5° C. and 20° C. overnight with 700 RPM stirring. The formulations were filtered through a 0.2 μm PTFE membrane and analyzed by HPLC. The results are shown in Table 10.

TABLE 10
Solubility of naloxone HCl at 20° C. and −5° C.

| | 20° C. solubility | −5° C. solubility |
|---|---|---|
| Composition | (mg/mL) | (mg/mL) |
| 20% w/v PG | 70.6 | 25.9 |
| 0.2% w/v EDTA-Na₂ | | |
| 20% w/v PG | 69.5 | 44.2 |
| 0.1% w/v EDTA-Na₂ | | |
| 20% w/v PG | 71.3 | 30.7 |
| 0% w/v EDTA-Na₂ | | |
| 10% w/v PG | 70.5 | Not determined |
| 0.1% w/v EDTA-Na₂ | | |
| 10% w/v PG | 71.4 | |
| 0% w/v EDTA-Na₂ | | |
| 5% w/v PG | 70.3 | |
| 0.1% w/v EDTA-Na₂ | | |
| 5% w/v PG | 71.2 | |
| 0% w/v EDTA-Na₂ | | |

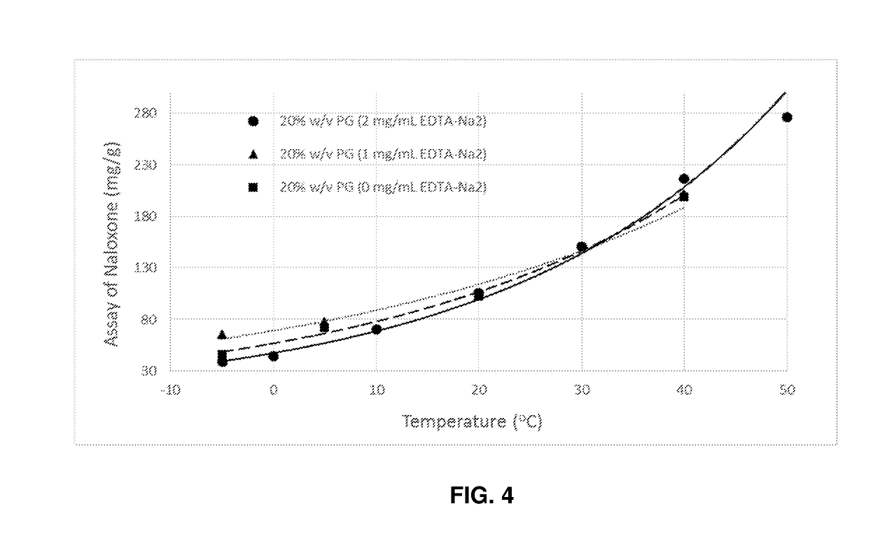

Solubility results indicate that: At lower temperatures, the solubility of naloxone is greatest in media consisting of 20% w/v PG and 0.1% w/v EDTA-Na₂ (FIG. 4, see also Table 9). By contrast, formulations containing 0.2% w/v EDTA-Na₂ exhibited the lowest solubility, opposite the findings with solutions held above 30° C. (data not shown).

There is little difference in the 20° C. solubility values of naloxone in all formulations examined (Table 9). The 20° C. solubility of these formulations are all slightly lower than the 20° C. solubility of naloxone HCl without PG at 0.2% w/v EDTA-Na₂ (i.e., 84 mg/mL).

To test the stability of the formulations over extended time, triplicate samples of the various formulations were left at 4° C. for extended periods and examined visually for precipitation at 7, 14, 24, 35, and 53 days. One of every three was spiked with silica gel to facilitate precipitation. The results are shown in Table 11 below.

TABLE 11
Time to precipitation at 4° C. with different concentrations
of EDTA and PG

| Composition | | Evidence of naloxone HCl precipitation | | | | |
|---|---|---|---|---|---|---|
| EDTA-Na₂ | PG | After 7 | After 14 | After 24 | After 35 | After 53 |
| (% w/v) | (% w/v) | days | days | days | days | days |
| 0.1 | 20 | No | No | No | No | No |
| | | No | No | No | Yes | Yes |
| | | No* | No* | Slight* | Yes* | Yes* |
| 0 | 20 | No | No | No | No | No |
| | | No | No | No | No | No |
| | | No* | No* | No* | No* | No* |
| 0.1 | 10 | No | No | No | No | No |
| | | No | No | No | No | No |
| | | No* | No* | No* | No* | Slight* |
| 0 | 10 | No | No | No | No | No |
| | | No | No | No | No | No |
| | | No* | No* | No* | No* | No* |
| 0.1 | 5 | No | No | No | No | No |
| | | No | No | No | No | No |
| | | No* | No* | No* | No* | No* |
| 0 | 5 | No | No | No | No | No |
| | | No | No | No | No | No |
| | | No* | No* | No* | No* | No* |

*Sample spiked with silica gel

From these results it can be seen reducing the amount of dissolved EDTA-Na₂ and lowering the concentration of PG cosolvent achieved a formulation that does not generate a precipitate, for any of the solutions tested up to 3 freeze/thaw cycles. The freezing points of the formulations containing 0%, 0.1%, and 0.2% (w/v) EDTA-Na₂ did not vary significantly. A 1 μL aliquot of 1 N HCl or NaOH was able to move the pH of a 100 mL sample of the 0% EDTA-Na₂ solution by 2 to 3 pH units.

To test the freezing points of these formulations, solutions were cooled to 0° C. and held there for 10 minutes, before gradual cooling to −20° C. at a rate of 0.3 C°/min. until the solution reached solidity. The formulations containing 20% (w/v) PG froze at approximately −15° C., the formulations containing 10% (w/v) PG at approximately −11° C., and the formulations containing 5% (w/v) PG at approximately −7° C. It was observed, however, that if the 20% (w/v) PG formulation was brought quickly to −10° C. (i.e., was not held for 10 min at 0° C. in the course of the freezing process) and then adjusted to −20° C. at a rate of 0.3 C°/min., then the freezing temperature ranged from −10° C. to −15° C.

### Example 5: Effects of Sorbitol

To test the effects of sorbitol addition on the freezing points of the solutions tested in Examples 1-4, the solution of Table 12 was prepared.

TABLE 12
Naloxone solution with 4% (w/v) sorbitol

| Components | Amount | |
|---|---|---|
| Naloxone | Saturation | |
| NaCl | 741 | mg |
| EDTA-Na | 99 | mg |
| BZK (50% in water) | 10 | mg |
| Propylene Glycol (20 g) | 19.23 | mL |
| Sorbitol | 4.002 | g |

| | |
|---|---|
| Purified Water | qs to 100 mL |
| pH | Adjust if necessary |

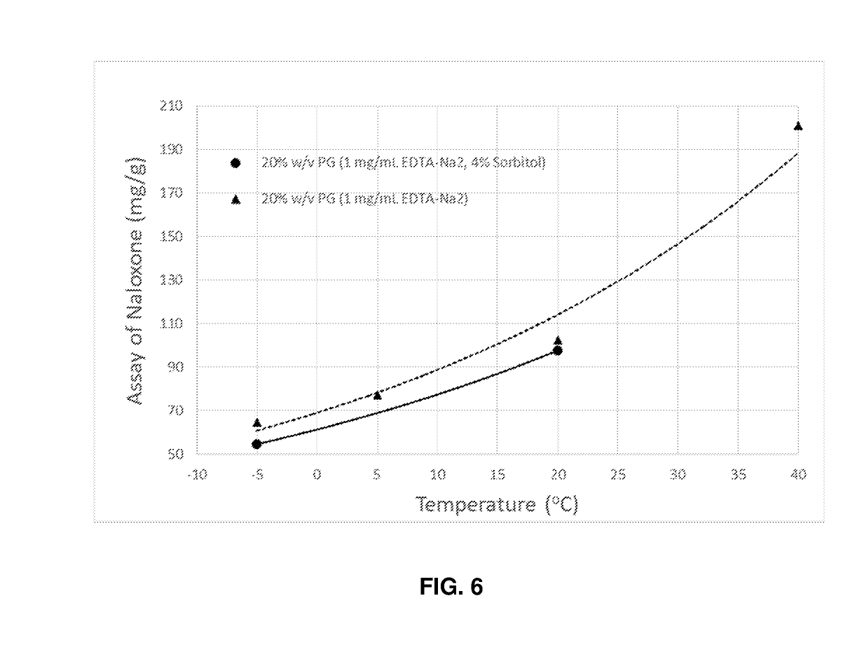

The solution was cooled to 0° C. with agitation at 700 RPM. After a 15 min. hold at 0° C., the solution was gradually cooled to −15° C. at a rate of 0.3° C./min. with continued agitation. As can be seen in FIG. 6, the addition of sorbitol had no significant effect on freezing temperature beyond that seen with PG. This solution showed no precipitation on thawing.

### Example 6: Pharmacokinetics

A phase I, single dose, open label, randomized, three-period crossover study is performed to compare the pharmacokinetics, safety, and tolerability of naloxone administration using the solutions described herein in healthy adults. The study compares the pharmacokinetics of naloxone when the solutions of the present disclosure (at 20 mg/mL and 40 mg/mL dosage strengths) are administered, compared to the currently FDA approved NARCAN products at the same dosage strengths.

The primary outcome measurements are: (1) plasma concentration time profiles and “area under the curve” (AUC); (2) maximum serum concentration (Cₘₐₓ); (3) time to maximum serum concentration (Tₘₐₓ); (4) elimination rate constant (Kₑₗ); and (5) terminal half-life (t_{1/2}).

Blood is collected in sodium heparin containing tubes for naloxone PK prior to dosing and 2.5, 5, 10, 15, 20, 30, 45, 60, 120, 180, 240, 300, 360, 480, and 720 minutes after the start of study drug administration. Plasma is separated from whole blood and stored frozen at <−20° C. until assayed. Naloxone plasma concentrations are determined by liquid chromatography with tandem mass spectrometry. Conjugated naloxone plasma concentrations may also be determined.

The secondary outcome measurements are: (1) number of subjects with adverse effects; (2) physical examination of subjects; (3) vital signs; and (4) electrocardiograms. Heart rate, blood pressure, and respiration rate are recorded before naloxone dosing and at approximately 30, 60, 120, and 480 minutes after dosing. A 12-lead ECG is obtained prior to and approximately 60 and 480 minutes after each naloxone dose. ECG and vital signs are measured within the 10 minute period before the nominal time for blood collections. Adverse events (AEs) are recorded from the start of study drug administration until clinic discharge. AEs are recorded relative to each dosing session to attempt to establish a relationship between the AE and type of naloxone dose administered. An examination of the nasal passage is conducted at Day −1 to establish eligibility and at pre-dose, 5 minutes, 30 minutes, 60 minutes, 4 hours, and 24 hours post naloxone administration to evaluate evidence of irritation to the nasal mucosa.

The data analysis plan examines non-compartmental PK parameters including Cₘₐₓ, Tₘₐₓ, AUC_{0-∞}, AUC₀₋ₜ, t_{1/2}, λ_{z}, and apparent clearance (CL/F). Pharmacokinetic parameters (Cₘₐₓ, Tₘₐₓ, and AUCs) for IN naloxone are compared with those for the reference IN naloxone. Tₘₐₓ is measured from the time of administration (spraying into the nasal cavity). Dose adjusted values for AUCs and Cₘₐₓ are calculated. The relative extent of intranasal absorption (IN versus reference IN) is estimated from the dose-corrected AUCs. Within an ANOVA framework, comparisons of In-transformed PK parameters (Cₘₐₓ and AUC) for intranasal versus the reference IN naloxone treatments are performed. The 90% confidence intervals for the ratio (IN/reference IN) of the geometric least squares means of AUC and Cₘₐₓ parameters are constructed for comparison of each treatment. These 90% confidence intervals are obtained by exponentiation of the 90% confidence intervals for the difference between the least squares means based upon an In scale.

AEs are coded using the most recent version of the Medical Dictionary for Regulatory Activities (MedDRA) preferred terms and will be grouped by system, organ, class (SOC) designation. The severity, frequency, and relationship of AEs to study drug are presented by preferred term by SOC grouping. Separate summaries are provided for the study periods: after the administration of each dose of study drug up until the time of the next dose of study drug or clinic discharge. Listings of each individual AE including start date, stop date, severity, relationship, outcome, and duration are provided.

Vital signs, ECG, and clinical laboratory parameters are presented as summary statistics and changes from baseline (with baseline being the measurement prior to each dose).

### Example 6: Assay of Impurities During Storage

Table 12 below shows the percentage area of the total impurities detected by HPLC assay of naloxone HCl. The results are calculated by subtracting the % area of the main naloxone peak from 100% (for two individual samples). There is a proportional relationship between the storage temperature and the total % area of impurities detected by HPLC across all formulations.

The % area of the total impurities for samples stored at 5° C. and 25° C./60% RH at T=0 are higher than at T=1 for 40 mg/mL naloxone HCl formulations containing 1 mg/mL Na₂.EDTA/100 mg/mL ultra-refined PG or 2 mg/mL Na₂.EDTA/0 mg/mL PG. The formulations were stored unprotected from light at room temperature and not analyzed until approximately 2 weeks after formulation preparation. A 40 mg/mL naloxone HCl formulation containing 1 mg/mL Na₂.EDTA/100 mg/mL SIGMA-ALDRICH PG, however, displayed lower amounts of total impurities (0.26% area) at T=0 while stored under the same conditions and analyzed after a comparable time frame. Notably, the formulation containing SIGMA-ALDRICH propylene glycol showed an increase in % area of total impurities detected across all temperatures after only 1 month stability storage (Table 13).

TABLE 13
Total impurities (% area), average of 2 HPLC injections

| EDTA | PG | T = 0 | T = 1 month | | | |
|---|---|---|---|---|---|---|
| (mg/mL) | (mg/mL) | 25° C. | 5° C. | 25° C. | 40° C. | 50° C. |
| 1 | 100* | 0.39 | 0.26 | 0.27 | 0.47 | 0.66 |
| 1 | 50* | 0.39 | 0.25 | 0.28 | 0.48 | 0.66 |
| 1 | 0 | 0.38 | 0.25 | 0.31 | 0.51 | 0.69 |
| 2 | 0 | 0.38 | 0.25 | 0.29 | 0.47 | 0.69 |
| 1 | 100^{‡} | 0.26 | 0.40 | 0.41 | 0.67 | 0.87 |
| | | | T = 2 months | | | |
| 1 | 100* | | 0.41 | 0.48 | 0.76 | 1.09 |
| 1 | 50* | | 0.40 | 0.47 | 0.78 | 1.09 |
| 1 | 0 | | 0.43 | 0.50 | 0.79 | 1.13 |
| 2 | 0 | | 0.42 | 0.48 | 0.75 | 1.10 |
| | | | T = 3 months | | | |
| 1 | 100* | | 0.32 | 0.37 | 0.82 | 1.15 |
| 1 | 50* | | 0.32 | 0.48 | 0.88 | 1.24 |
| 1 | 0 | | 0.30 | 0.42 | 0.90 | 1.24 |
| 2 | 0 | | 0.31 | 0.39 | 0.90 | 1.22 |

*Ultra refined USP grade PG (CRODA)
^{‡}USP grade PG (SIGMA-ALDRICH)

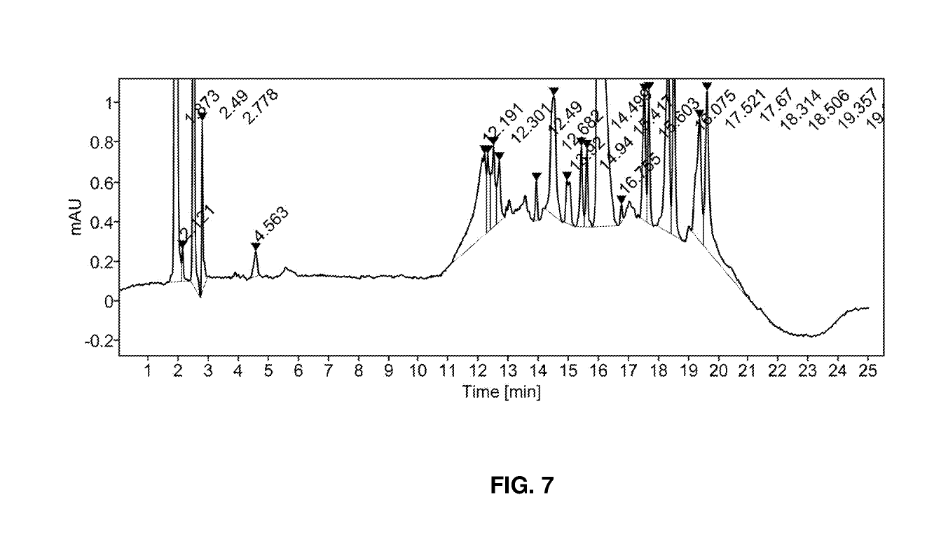

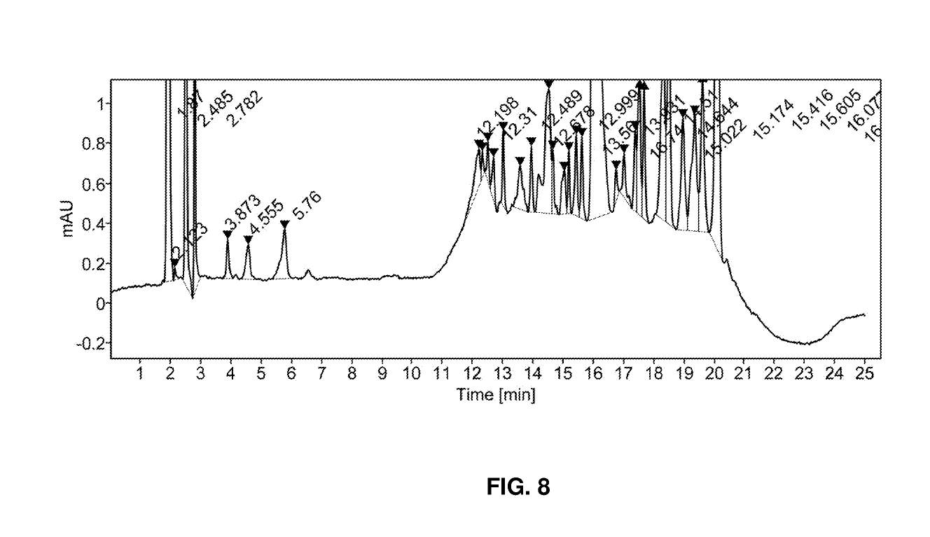

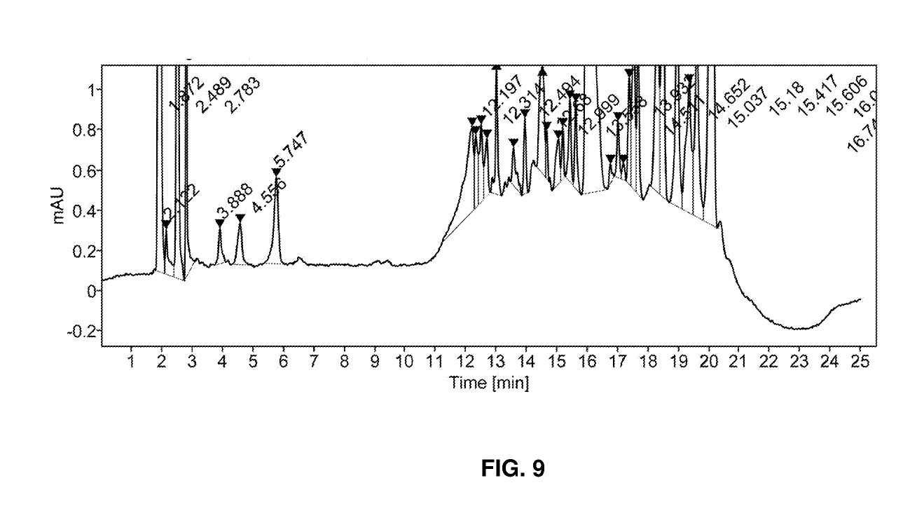

Detailed analysis of all HPLC peak impurities detected during the assay of naloxone HCl are shown in the Tables 14-18 below. New impurities were observed with relative retention times greater than 1 after 3 months storage. New peaks were observed with approximate retention times of retention times of about 3.9 minutes, 5.7 minutes, 6.5 minutes, and 20 minutes after 6 and 10 days storage (FIGS. 7-9). New peaks were observed with approximate retention times of 7.1 minutes (relative retention time, “RRT” 1.09) and 7.6 minutes (RRT 1.17) after 3 months storage. These peaks are not present for formulations stored at 5° C. and 25° C. In addition, the intensity of these peaks increased with storage time, and are observed even after one month storage. The NARCAN control formulation (2 mg EDTA/0 mg PG) displayed a relatively smaller impurity peak (and perhaps another co-eluting with the main naloxone peak).

TABLE 14
Total impurities (% area), average of 2 HPLC injections for 40 mg/mL
naloxone HCl solution containing 1 mg/mL EDTA and 100 mg/mL ultra-
refined propylene glycol

| | RRT | T = 0 | T = 1 month | | | |
|---|---|---|---|---|---|---|
| RT [min] | (approx.) | 25° C. | 5° C. | 25° C. | 40° C. | 50° C. |
| 1.826-1.853 | 0.28 | ND | ND | ND | ND | ND |
| 1.912 | 0.30 | ND | ND | ND | ND | ND |
| 1.988-2.259 | 0.31 | 0.14 | 0.04 | 0.03 | 0.03 | 0.03 |
| 2.111 | 0.33 | 0.01 | ND | ND | ND | ND |
| 2.270-2.334 | 0.35 | 0.09 | 0.07 | 0.07 | 0.09 | 0.15 |
| 2.413-2.47 | 0.37 | ND | ND | ND | ND | ND |
| 2.605-2.649 | 0.40 | ND | ND | ND | ND | ND |
| 2.747-2.772 | 0.42 | 0.01 | 0.01 | 0.01 | 0.01 | 0.01 |
| 2.791 | 0.43 | ND | ND | ND | 0.02 | 0.04 |
| 3.018-3.026 | 0.47 | ND | ND | ND | ND | ND |
| 3.219-3.236 | 0.50 | 0.01 | 0.01 | 0.01 | 0.02 | 0.06 |
| 3.344-3.379 | 0.52 | ND | ND | ND | ND | ND |
| 3.422-3.734 | 0.53 | ND | ND | ND | 0.01 | 0.01 |
| 3.802-3.927 | 0.59 | 0.04 | 0.04 | 0.04 | 0.04 | 0.05 |
| 4.012-4.108 | 0.62 | 0.02 | 0.02 | 0.02 | 0.06 | 0.06 |
| 4.577 | 0.71 | 0.01 | 0.01 | 0.01 | 0.02 | 0.01 |
| 4.729-4.868 | 0.73 | 0.02 | 0.02 | 0.02 | 0.02 | 0.02 |
| 5.031-5.256 | 0.78 | ND | 0.01 | 0.01 | 0.02 | 0.04 |
| 5.334-5.473 | 0.82 | 0.01 | ND | ND | ND | ND |
| 6.468-6.686 | 1.00 | 99.61 | 99.74 | 99.73 | 99.53 | 99.34 |
| 7.100-7.227 | 1.10 | ND | ND | ND | 0.03 | 0.03 |
| 7.572-7.732 | 1.17 | ND | ND | ND | 0.01 | 0.03 |
| 8.581-8.857 | 1.33 | 0.01 | 0.01 | 0.01 | 0.01 | 0.01 |
| 9.216-9.234 | 1.42 | 0.02 | 0.02 | 0.02 | 0.02 | 0.04 |
| 9.617-9.840 | 1.50 | ND | ND | ND | ND | ND |
| 11.053-11.439 | 1.71 | 0.01 | 0.01 | 0.02 | 0.08 | 0.10 |
| 11.852-11.925 | 1.83 | ND | ND | ND | ND | ND |
| | | | T = 2 months | | | |
| 1.826-1.853 | 0.28 | | ND | ND | ND | ND |
| 1.912 | 0.30 | | ND | ND | ND | ND |
| 1.988-2.259 | 0.31 | | 0.16 | 0.17 | 0.17 | 0.19 |
| 2.111 | 0.33 | | ND | ND | ND | ND |
| 2.270-2.334 | 0.35 | | 0.10 | 0.12 | 0.18 | 0.26 |
| 2.413-2.47 | 0.37 | | ND | ND | ND | ND |
| 2.605-2.649 | 0.40 | | ND | ND | ND | ND |
| 2.747-2.772 | 0.42 | | 0.01 | 0.01 | 0.04 | 0.08 |
| 2.791 | 0.43 | | 0.01 | ND | ND | ND |
| 3.018-3.026 | 0.47 | | ND | ND | ND | ND |
| 3.219-3.236 | 0.50 | | ND | 0.01 | 0.05 | 0.09 |
| 3.344-3.379 | 0.52 | | ND | ND | ND | ND |
| 3.422-3.734 | 0.53 | | ND | ND | ND | ND |
| 3.802-3.927 | 0.59 | | 0.04 | 0.04 | 0.04 | 0.05 |
| 4.012-4.108 | 0.62 | | 0.02 | 0.03 | 0.06 | 0.07 |
| 4.577 | 0.71 | | 0.02 | ND | ND | ND |
| 4.729-4.868 | 0.73 | | 0.01 | 0.02 | 0.02 | 0.02 |
| 5.031-5.256 | 0.78 | | 0.01 | ND | 0.02 | 0.05 |
| 5.334-5.473 | 0.82 | | ND | 0.01 | 0.01 | 0.01 |
| 6.468-6.686 | 1.00 | | 99.59 | 99.52 | 99.24 | 98.91 |
| 7.100-7.227 | 1.10 | | ND | ND | 0.03 | 0.06 |
| 7.572-7.732 | 1.17 | | ND | ND | 0.02 | 0.05 |
| 8.581-8.857 | 1.33 | | 0.01 | 0.01 | 0.01 | 0.01 |
| 9.216-9.234 | 1.42 | | 0.02 | 0.02 | 0.03 | 0.06 |
| 9.617-9.840 | 1.50 | | ND | ND | ND | ND |
| 11.053-11.439 | 1.71 | | 0.01 | 0.03 | 0.09 | 0.08 |
| 11.852-11.925 | 1.83 | | ND | ND | ND | ND |
| | | | T = 3 months | | | |
| 1.826-1.853 | 0.28 | | ND | ND | 0.00 | 0.01 |
| 1.912 | 0.30 | | 0.02 | 0.02 | 0.01 | 0.00 |
| 1.988-2.259 | 0.31 | | 0.02 | 0.01 | 0.02 | 0.04 |
| 2.111 | 0.33 | | ND | 0.04 | 0.00 | 0.00 |
| 2.270-2.334 | 0.35 | | 0.09 | 0.08 | 0.15 | 0.27 |
| 2.413-2.47 | 0.37 | | 0.00 | ND | 0.01 | 0.01 |
| 2.605-2.649 | 0.40 | | 0.01 | 0.00 | 0.02 | 0.03 |
| 2.747-2.772 | 0.42 | | 0.01 | 0.02 | 0.04 | 0.08 |
| 2.791 | 0.43 | | ND | ND | 0.00 | 0.00 |
| 3.018-3.026 | 0.47 | | ND | 0.00 | 0.00 | 0.00 |
| 3.219-3.236 | 0.50 | | 0.01 | 0.01 | 0.07 | 0.05 |
| 3.344-3.379 | 0.52 | | 0.01 | 0.01 | 0.00 | 0.01 |
| 3.422-3.734 | 0.53 | | ND | ND | ND | ND |
| 3.802-3.927 | 0.59 | | 0.04 | 0.04 | 0.05 | 0.06 |
| 4.012-4.108 | 0.62 | | 0.02 | 0.03 | 0.06 | 0.06 |
| 4.577 | 0.71 | | 0.01 | ND | 0.00 | 0.01 |
| 4.729-4.868 | 0.73 | | 0.02 | 0.02 | 0.02 | 0.02 |
| 5.031-5.256 | 0.78 | | 0.00 | 0.00 | 0.03 | 0.05 |
| 5.334-5.473 | 0.82 | | 0.01 | 0.01 | 0.01 | 0.01 |
| 6.468-6.686 | 1.00 | | 99.68 | 99.63 | 99.18 | 98.85 |
| 7.100-7.227 | 1.10 | | ND | ND | 0.03 | 0.07 |
| 7.572-7.732 | 1.17 | | ND | 0.00 | 0.03 | 0.09 |
| 8.581-8.857 | 1.33 | | 0.01 | 0.01 | 0.01 | 0.00 |
| 9.216-9.234 | 1.42 | | 0.02 | 0.02 | 0.04 | 0.08 |
| 9.617-9.840 | 1.50 | | 0.00 | 0.00 | 0.00 | 0.00 |
| 11.053-11.439 | 1.71 | | 0.01 | 0.06 | 0.13 | 0.11 |
| 11.852-11.925 | 1.83 | | ND | ND | ND | 0.01 |

ND = no peak detected

TABLE 15
Total impurities (% area), average of 2 HPLC injections for 40 mg/mL
naloxone HCl solution containing 1 mg/mL EDTA and 50 mg/mL ultra-
refined propylene glycol

| | RRT | T = 0 | T = 1 month | | | |
|---|---|---|---|---|---|---|
| RT [min] | (approx.) | 25° C. | 5° C. | 25° C. | 40° C. | 50° C. |
| 1.822-1.929 | 0.28 | ND | ND | ND | ND | ND |
| 1.963-1.988 | 0.30 | 0.14 | 0.03 | 0.03 | 0.04 | 0.03 |
| 2.109 | 0.33 | 0.01 | ND | ND | ND | ND |
| 2.260-2.426 | 0.35 | 0.09 | 0.07 | 0.07 | 0.10 | 0.15 |
| 2.4052.538 | 0.37 | ND | ND | ND | ND | ND |
| 2.712-2.809 | 0.42 | 0.01 | 0.01 | 0.01 | 0.01 | 0.01 |
| 2.724-2.809 | 0.42 | ND | ND | ND | 0.02 | 0.04 |
| 3.020 | 0.47 | ND | ND | ND | ND | ND |
| 3.012-3.228 | 0.46 | ND | ND | ND | ND | ND |
| 3.240-3.376 | 0.50 | 0.01 | 0.01 | 0.01 | 0.02 | 0.05 |
| 3.213-3.406 | 0.50 | ND | ND | ND | 0.01 | 0.01 |
| 3.735 | 0.57 | ND | ND | ND | ND | ND |
| 3.724-3.931 | 0.57 | 0.04 | 0.04 | 0.04 | 0.04 | 0.05 |
| 4.008-4.102 | 0.62 | 0.02 | 0.01 | 0.02 | 0.06 | 0.06 |
| 4.576 | 0.71 | 0.01 | 0.02 | 0.01 | 0.02 | ND |
| 4.741-4.864 | 0.73 | 0.02 | 0.02 | 0.02 | 0.02 | 0.02 |
| 4.970-5.108 | 0.77 | ND | ND | ND | ND | ND |
| 5.250 | 0.81 | ND | 0.01 | 0.01 | 0.02 | 0.05 |
| 5.293-5.464 | 0.82 | 0.01 | ND | ND | ND | ND |
| 6.483-6.673 | 1.00 | 99.61 | 99.75 | 99.72 | 99.52 | 99.34 |
| 7.091-7.108 | 1.09 | ND | ND | ND | 0.02 | 0.03 |
| 7.574-7.710 | 1.17 | ND | ND | ND | 0.01 | 0.02 |
| 8.521-8.829 | 1.31 | 0.01 | 0.01 | 0.01 | 0.01 | 0.01 |
| 9.119-9.360 | 1.41 | 0.02 | 0.02 | 0.02 | 0.03 | 0.05 |
| 9.594-9.873 | 1.48 | ND | ND | ND | ND | ND |
| 10.957-11.418 | 1.69 | 0.01 | 0.01 | 0.03 | 0.08 | 0.10 |
| | | | T = 2 months | | | |
| 1.822-1.929 | 0.28 | | ND | ND | ND | ND |
| 1.963-1.988 | 0.30 | | 0.15 | 0.15 | 0.16 | 0.17 |
| 2.109 | 0.33 | | ND | ND | ND | ND |
| 2.260-2.426 | 0.35 | | 0.10 | 0.12 | 0.19 | 0.30 |
| 2.4052.538 | 0.37 | | ND | ND | ND | ND |
| 2.712-2.809 | 0.42 | | ND | ND | ND | ND |
| 2.724-2.809 | 0.42 | | 0.01 | 0.01 | 0.04 | 0.08 |
| 3.020 | 0.47 | | ND | ND | ND | ND |
| 3.012-3.228 | 0.46 | | ND | ND | ND | ND |
| 3.240-3.376 | 0.50 | | 0.01 | 0.01 | 0.05 | 0.09 |
| 3.213-3.406 | 0.50 | | ND | ND | ND | ND |
| 3.735 | 0.57 | | ND | ND | ND | ND |
| 3.724-3.931 | 0.57 | | 0.04 | 0.04 | 0.04 | 0.06 |
| 4.008-4.102 | 0.62 | | 0.02 | 0.03 | 0.07 | 0.07 |
| 4.576 | 0.71 | | ND | ND | ND | ND |
| 4.741-4.864 | 0.73 | | 0.02 | 0.02 | 0.02 | 0.02 |
| 4.970-5.108 | 0.77 | | ND | 0.01 | ND | 0.06 |
| 5.250 | 0.81 | | 0.01 | ND | 0.02 | ND |
| 5.293-5.464 | 0.82 | | 0.01 | 0.01 | 0.01 | 0.01 |
| 6.483-6.673 | 1.00 | | 99.60 | 99.53 | 99.22 | 98.91 |
| 7.091-7.108 | 1.09 | | ND | ND | 0.02 | 0.05 |
| 7.574-7.710 | 1.17 | | ND | ND | 0.02 | 0.04 |
| 8.521-8.829 | 1.31 | | 0.01 | 0.01 | 0.01 | 0.01 |
| 9.119-9.360 | 1.41 | | 0.02 | 0.02 | 0.03 | 0.07 |
| 9.594-9.873 | 1.48 | | ND | ND | ND | ND |
| 10.957-11.418 | 1.69 | | 0.01 | 0.04 | 0.10 | 0.09 |
| | | | T = 3 months | | | |
| 1.822-1.929 | 0.28 | | 0.01 | 0.01 | 0.00 | 0.02 |
| 1.963-1.988 | 0.30 | | 0.02 | 0.02 | 0.03 | 0.05 |
| 2.109 | 0.33 | | 0.00 | 0.00 | 0.00 | 0.00 |
| 2.260-2.426 | 0.35 | | 0.09 | 0.09 | 0.18 | 0.31 |
| 2.4052.538 | 0.37 | | ND | 0.00 | 0.01 | 0.01 |
| 2.712-2.809 | 0.42 | | ND | ND | 0.00 | 0.00 |
| 2.724-2.809 | 0.42 | | 0.02 | 0.02 | 0.05 | 0.09 |
| 3.020 | 0.47 | | ND | 0.00 | 0.00 | 0.01 |
| 3.012-3.228 | 0.46 | | 0.01 | 0.01 | 0.01 | 0.10 |
| 3.240-3.376 | 0.50 | | 0.01 | 0.01 | 0.00 | 0.00 |
| 3.213-3.406 | 0.50 | | 0.00 | 0.00 | 0.06 | 0.01 |
| 3.735 | 0.57 | | ND | 0.04 | 0.05 | 0.07 |
| 3.724-3.931 | 0.57 | | 0.04 | 0.03 | 0.07 | 0.07 |
| 4.008-4.102 | 0.62 | | 0.00 | 0.00 | 0.01 | 0.01 |
| 4.576 | 0.71 | | 0.00 | 0.00 | 0.00 | 0.00 |
| 4.741-4.864 | 0.73 | | 0.02 | 0.02 | 0.02 | 0.06 |
| 4.970-5.108 | 0.77 | | 0.01 | 0.00 | 0.03 | 0.06 |
| 5.250 | 0.81 | | 0.00 | 0.00 | 0.00 | 0.00 |
| 5.293-5.464 | 0.82 | | 0.01 | 0.01 | 0.01 | 0.00 |
| 6.483-6.673 | 1.00 | | 99.68 | 99.62 | 99.12 | 98.76 |
| 7.091-7.108 | 1.09 | | 0.00 | 0.00 | 0.02 | 0.05 |
| 7.574-7.710 | 1.17 | | 0.00 | 0.00 | 0.02 | 0.06 |
| 8.521-8.829 | 1.31 | | 0.01 | 0.01 | 0.01 | 0.00 |
| 9.119-9.360 | 1.41 | | 0.01 | 0.02 | 0.04 | 0.09 |
| 9.594-9.873 | 1.48 | | 0.01 | 0.00 | 0.00 | 0.00 |
| 10.957-11.418 | 1.69 | | 0.01 | 0.07 | 0.14 | 0.12 |

ND = no peak detected

TABLE 16
Total impurities (% area), average of 2 HPLC injections for 40 mg/mL
naloxone HCl solution containing 1 mg/mL EDTA and 0 mg/mL
propylene glycol

| | RRT | T = 0 | T = 1 month | | | |
|---|---|---|---|---|---|---|
| RT [min] | (approx.) | 25° C. | 5° C. | 25° C. | 40° C. | 50° C. |
| 1.822 | 0.28 | ND | ND | ND | ND | ND |
| 1.962-1.988 | 0.30 | 0.14 | 0.03 | 0.03 | 0.03 | 0.04 |
| 2.108-2.262 | 0.32 | 0.01 | ND | ND | 0.11 | ND |
| 2.332-2.623 | 0.36 | 0.09 | 0.07 | 0.07 | 0.01 | 0.18 |
| 2.643-2.768 | 0.41 | ND | ND | ND | ND | 0.01 |
| 2.768 | 0.43 | 0.01 | 0.01 | 0.02 | 0.02 | 0.04 |
| 3.003 | 0.46 | ND | ND | ND | ND | ND |
| 3.016-3.21 | 0.46 | ND | ND | ND | ND | ND |
| 3.218-3.294 | 0.50 | ND | ND | ND | ND | 0.05 |
| 3.277-3.377 | 0.51 | 0.01 | 0.01 | 0.01 | 0.02 | 0.01 |
| 3.699-3.799 | 0.57 | 0.04 | 0.04 | 0.04 | 0.01 | 0.05 |
| 3.890-4.008 | 0.60 | 0.02 | 0.02 | 0.03 | 0.04 | 0.07 |
| 4.573 | 0.70 | 0.01 | 0.01 | 0.02 | 0.07 | ND |
| 4.704-4.864 | 0.73 | 0.02 | 0.02 | 0.02 | 0.01 | 0.02 |
| 4.998-5.256 | 0.77 | 0.00 | 0.01 | 0.01 | 0.02 | 0.05 |
| 5.261-5.460 | 0.81 | 0.01 | ND | ND | 0.02 | ND |
| 6.487-6.678 | 1.00 | 99.62 | 99.75 | 99.69 | 99.49 | 99.31 |
| 7.337-7.668 | 1.13 | ND | ND | ND | ND | 0.01 |
| 8.226 | 1.27 | ND | ND | ND | ND | ND |
| 8.456-8.828 | 1.30 | 0.01 | 0.01 | 0.01 | 0.02 | 0.01 |
| 9.332 | 1.44 | 0.02 | 0.02 | 0.02 | 0.03 | 0.05 |
| 9.719-9.883 | 1.50 | ND | ND | ND | ND | ND |
| 10.716-11.028 | 1.65 | 0.01 | 0.01 | 0.04 | 0.10 | 0.11 |
| | | | T = 2 months | | | |
| 1.822 | 0.28 | | ND | ND | ND | ND |
| 1.962-1.988 | 0.30 | | 0.16 | 0.16 | 0.17 | 0.18 |
| 2.108-2.262 | 0.32 | | ND | ND | ND | ND |
| 2.332-2.623 | 0.36 | | 0.10 | 0.12 | 0.21 | 0.35 |
| 2.643-2.768 | 0.41 | | 0.01 | 0.01 | 0.04 | 0.09 |
| 2.768 | 0.43 | | ND | ND | ND | ND |
| 3.003 | 0.46 | | ND | ND | ND | ND |
| 3.016-3.21 | 0.46 | | ND | ND | ND | ND |
| 3.218-3.294 | 0.50 | | 0.01 | 0.01 | 0.05 | 0.09 |
| 3.277-3.377 | 0.51 | | 0.04 | ND | ND | ND |
| 3.699-3.799 | 0.57 | | ND | 0.04 | 0.04 | 0.06 |
| 3.890-4.008 | 0.60 | | 0.02 | 0.04 | 0.07 | 0.07 |
| 4.573 | 0.70 | | ND | 0.02 | ND | ND |
| 4.704-4.864 | 0.73 | | 0.02 | ND | 0.02 | 0.02 |
| 4.998-5.256 | 0.77 | | ND | 0.01 | 0.03 | 0.07 |
| 5.261-5.460 | 0.81 | | 0.01 | 0.01 | 0.01 | 0.01 |
| 6.487-6.678 | 1.00 | | 99.57 | 99.50 | 99.21 | 98.87 |
| 7.337-7.668 | 1.13 | | ND | ND | 0.01 | 0.03 |
| 8.226 | 1.27 | | ND | ND | ND | ND |
| 8.456-8.828 | 1.30 | | 0.01 | 0.01 | 0.02 | 0.02 |
| 9.332 | 1.44 | | 0.02 | 0.02 | 0.03 | 0.07 |
| 9.719-9.883 | 1.50 | | ND | ND | ND | ND |
| 10.716-11.028 | 1.65 | | 0.01 | 0.05 | 0.10 | 0.09 |
| | | | T = 3 months | | | |
| 1.822 | 0.28 | | ND | ND | 0.00 | 0.02 |
| 1.962-1.988 | 0.30 | | 0.03 | 0.03 | 0.03 | 0.06 |
| 2.108-2.262 | 0.32 | | ND | ND | ND | ND |
| 2.332-2.623 | 0.36 | | 0.08 | 0.11 | 0.20 | 0.27 |
| 2.643-2.768 | 0.41 | | 0.01 | 0.03 | 0.03 | 0.05 |
| 2.768 | 0.43 | | 0.00 | 0.00 | 0.05 | 0.00 |
| 3.003 | 0.46 | | 0.01 | 0.00 | 0.00 | 0.09 |
| 3.016-3.21 | 0.46 | | ND | 0.00 | 0.04 | 0.01 |
| 3.218-3.294 | 0.50 | | 0.01 | 0.01 | 0.03 | 0.10 |
| 3.277-3.377 | 0.51 | | 0.00 | 0.00 | 0.00 | 0.01 |
| 3.699-3.799 | 0.57 | | 0.04 | 0.04 | 0.05 | 0.06 |
| 3.890-4.008 | 0.60 | | 0.02 | 0.04 | 0.08 | 0.06 |
| 4.573 | 0.70 | | 0.00 | 0.00 | 0.01 | 0.00 |
| 4.704-4.864 | 0.73 | | 0.02 | 0.02 | 0.02 | 0.01 |
| 4.998-5.256 | 0.77 | | 0.00 | 0.00 | 0.04 | 0.06 |
| 5.261-5.460 | 0.81 | | 0.01 | 0.00 | 0.02 | 0.03 |
| 6.487-6.678 | 1.00 | | 99.70 | 99.58 | 99.10 | 98.76 |
| 7.337-7.668 | 1.13 | | 0.00 | 0.00 | 0.01 | 0.03 |
| 8.226 | 1.27 | | ND | ND | 0.01 | 0.00 |
| 8.456-8.828 | 1.30 | | 0.01 | 0.01 | 0.01 | 0.00 |
| 9.332 | 1.44 | | 0.02 | 0.02 | 0.05 | 0.09 |
| 9.719-9.883 | 1.50 | | 0.00 | 0.00 | 0.00 | 0.00 |
| 10.716-11.028 | 1.65 | | 0.02 | 0.08 | 0.15 | 0.11 |

TABLE 17
Total impurities (% area), average of 2 HPLC injections for 40 mg/mL
naloxone HCl solution containing 2 mg/mL EDTA and 0 mg/mL
propylene glycol

| | RRT | T = 0 | T = 1 month | | | |
|---|---|---|---|---|---|---|
| RT [min] | (approx.) | 25° C. | 5° C. | 25° C. | 40° C. | 50° C. |
| 1.822 | 0.28 | ND | ND | ND | ND | ND |
| 1.961-1.985 | 0.30 | 0.13 | 0.03 | 0.03 | 0.03 | 0.04 |
| 1.976-2.109 | 0.31 | 0.01 | ND | ND | ND | ND |
| 2.252-2.333 | 0.35 | 0.09 | 0.07 | 0.07 | 0.11 | 0.18 |
| 2.403-2.606 | 0.37 | ND | ND | ND | ND | ND |
| 2.637-2.772 | 0.41 | 0.01 | 0.01 | 0.01 | 0.01 | 0.01 |
| 2.714-2.783 | 0.42 | ND | ND | ND | 0.02 | 0.04 |
| 3.013-3.023 | 0.47 | ND | ND | ND | ND | ND |
| 3.012-3.234 | 0.47 | ND | ND | ND | ND | ND |
| 3.289-3.382 | 0.51 | 0.01 | 0.01 | 0.01 | 0.02 | 0.05 |
| 3.413 | 0.53 | ND | ND | ND | 0.01 | 0.01 |
| 3.715-3.803 | 0.58 | 0.04 | 0.04 | 0.04 | 0.04 | 0.05 |
| 3.910-4.030 | 0.61 | 0.02 | 0.02 | 0.02 | 0.07 | 0.08 |
| 4.030 | 0.62 | ND | ND | ND | ND | ND |
| 4.563 | 0.71 | 0.01 | 0.01 | 0.01 | 0.02 | ND |
| 4.726-4.866 | 0.73 | 0.02 | 0.02 | 0.02 | 0.02 | 0.02 |
| 5.019-5.177 | 0.78 | ND | ND | ND | ND | 0.06 |
| 5.237 | 0.81 | ND | 0.01 | 0.01 | 0.02 | ND |
| 5.295-5.447 | 0.82 | 0.01 | ND | ND | ND | ND |
| 6.456-6.669 | 1.00 | 99.62 | 99.75 | 99.71 | 99.53 | 99.31 |
| 7.337-7.443 | 1.14 | ND | ND | ND | ND | ND |
| 7.695 | 1.19 | ND | ND | ND | ND | 0.01 |
| 8.226 | 1.27 | ND | ND | ND | ND | ND |
| 8.488-8.813 | 1.31 | 0.01 | 0.01 | 0.01 | 0.01 | 0.01 |
| 9.130-9.351 | 1.41 | 0.02 | 0.02 | 0.02 | 0.02 | 0.05 |
| 9.762-9.815 | 1.51 | ND | ND | ND | ND | ND |
| 10.975-11.376 | 1.70 | 0.01 | 0.01 | 0.03 | 0.10 | 0.11 |
| 11.883-11.902 | 1.84 | ND | ND | ND | ND | ND |
| | | | T = 2 months | | | |
| 1.822 | 0.28 | | ND | ND | ND | ND |
| 1.961-1.985 | 0.30 | | 0.16 | 0.16 | 0.16 | 0.18 |
| 1.976-2.109 | 0.31 | | ND | ND | ND | ND |
| 2.252-2.333 | 0.35 | | 0.11 | 0.13 | 0.19 | 0.32 |
| 2.403-2.606 | 0.37 | | ND | ND | ND | ND |
| 2.637-2.772 | 0.41 | | 0.01 | ND | 0.03 | 0.08 |
| 2.714-2.783 | 0.42 | | ND | 0.01 | ND | ND |
| 3.013-3.023 | 0.47 | | ND | ND | ND | ND |
| 3.012-3.234 | 0.47 | | ND | ND | ND | ND |
| 3.289-3.382 | 0.51 | | 0.01 | 0.01 | 0.05 | 0.09 |
| 3.413 | 0.53 | | ND | ND | ND | ND |
| 3.715-3.803 | 0.58 | | 0.04 | 0.04 | 0.04 | 0.06 |
| 3.910-4.030 | 0.61 | | 0.02 | 0.04 | 0.07 | 0.08 |
| 4.030 | 0.62 | | ND | ND | ND | ND |
| 4.563 | 0.71 | | ND | ND | ND | ND |
| 4.726-4.866 | 0.73 | | 0.02 | 0.02 | 0.02 | 0.02 |
| 5.019-5.177 | 0.78 | | ND | 0.01 | 0.02 | 0.07 |
| 5.237 | 0.81 | | 0.01 | 0.01 | 0.01 | 0.02 |
| 5.295-5.447 | 0.82 | | ND | ND | ND | ND |
| 6.456-6.669 | 1.00 | | 99.58 | 99.52 | 99.25 | 98.90 |
| 7.337-7.443 | 1.14 | | ND | ND | 0.01 | 0.02 |
| 7.695 | 1.19 | | ND | ND | ND | 0.01 |
| 8.226 | 1.27 | | ND | ND | ND | ND |
| 8.488-8.813 | 1.31 | | 0.01 | 0.01 | 0.02 | 0.02 |
| 9.130-9.351 | 1.41 | | 0.02 | 0.02 | 0.04 | 0.07 |
| 9.762-9.815 | 1.51 | | ND | ND | ND | 0.00 |
| 10.975-11.376 | 1.70 | | 0.01 | 0.05 | 0.10 | 0.09 |
| 11.883-11.902 | 1.84 | | ND | ND | ND | ND |
| | | | T = 3 months | | | |
| 1.822 | 0.28 | | ND | ND | 0.00 | 0.01 |
| 1.961-1.985 | 0.30 | | 0.02 | 0.00 | 0.03 | 0.05 |
| 1.976-2.109 | 0.31 | | 0.01 | 0.03 | 0.00 | 0.00 |
| 2.252-2.333 | 0.35 | | 0.08 | 0.09 | 0.20 | 0.34 |
| 2.403-2.606 | 0.37 | | 0.00 | 0.01 | 0.01 | 0.01 |
| 2.637-2.772 | 0.41 | | 0.02 | 0.03 | 0.03 | 0.04 |
| 2.714-2.783 | 0.42 | | 0.00 | 0.00 | 0.05 | 0.10 |
| 3.013-3.023 | 0.47 | | 0.00 | 0.00 | 0.00 | 0.01 |
| 3.012-3.234 | 0.47 | | 0.01 | 0.01 | 0.01 | 0.10 |
| 3.289-3.382 | 0.51 | | 0.01 | 0.01 | 0.06 | 0.01 |
| 3.413 | 0.53 | | 0.00 | 0.00 | 0.00 | 0.00 |
| 3.715-3.803 | 0.58 | | 0.04 | 0.04 | 0.05 | 0.07 |
| 3.910-4.030 | 0.61 | | 0.02 | 0.04 | 0.08 | 0.06 |
| 4.030 | 0.62 | | ND | ND | 0.01 | 0.00 |
| 4.563 | 0.71 | | 0.00 | 0.00 | 0.00 | 0.00 |
| 4.726-4.866 | 0.73 | | 0.02 | 0.02 | 0.02 | 0.01 |
| 5.019-5.177 | 0.78 | | 0.00 | 0.00 | 0.04 | 0.07 |
| 5.237 | 0.81 | | 0.00 | 0.00 | 0.00 | 0.00 |
| 5.295-5.447 | 0.82 | | 0.01 | 0.01 | 0.02 | 0.03 |
| 6.456-6.669 | 1.00 | | 99.69 | 99.61 | 99.10 | 98.78 |
| 7.337-7.443 | 1.14 | | 0.00 | 0.00 | 0.01 | 0.04 |
| 7.695 | 1.19 | | 0.00 | 0.00 | 0.00 | 0.00 |
| 8.226 | 1.27 | | ND | ND | 0.01 | 0.00 |
| 8.488-8.813 | 1.31 | | 0.01 | 0.01 | 0.01 | 0.00 |
| 9.130-9.351 | 1.41 | | 0.02 | 0.02 | 0.05 | 0.10 |
| 9.762-9.815 | 1.51 | | 0.00 | 0.00 | 0.00 | 0.00 |
| 10.975-11.376 | 1.70 | | 0.01 | 0.07 | 0.15 | 0.12 |
| 11.883-11.902 | 1.84 | | 0.00 | 0.00 | 0.00 | 0.01 |

ND = no peak detected

TABLE 18
Total impurities (% area), average of 2 HPLC injections for 40 mg/mL
naloxone HCl solution containing 1 mg/mL EDTA and 100 mg/mL
SIGMA-ALDRICH propylene glycol

| | RRT | T = 0 | T = 1 month | | | |
|---|---|---|---|---|---|---|
| RT [min] | (approx.) | 25° C. | 5° C. | 25° C. | 40° C. | 50° C. |
| 1.96 | 0.29 | 0.03 | 0.14 | 0.16 | 0.17 | 0.18 |
| 2.33 | 0.35 | 0.04 | 0.10 | 0.12 | 0.17 | 0.23 |
| 2.77 | 0.41 | ND | 0.01 | 0.01 | 0.03 | 0.04 |
| 3.24 | 0.48 | ND | 0.01 | 0.02 | 0.05 | 0.08 |
| 3.42 | 0.51 | 0.01 | ND | ND | ND | ND |
| 3.80 | 0.57 | 0.04 | 0.04 | 0.04 | 0.04 | 0.05 |
| 4.01 | 0.60 | 0.02 | 0.03 | 0.03 | 0.06 | 0.07 |
| 4.58 | 0.68 | 0.01 | 0.02 | 0.02 | 0.02 | 0.02 |
| 4.87 | 0.73 | 0.02 | ND | ND | 0.01 | ND |
| 5.26 | 0.79 | ND | ND | 0.01 | 0.01 | 0.03 |
| 5.47 | 0.82 | 0.01 | 0.01 | ND | ND | 0.01 |
| 6.69 | 1.00 | 99.74 | 99.60 | 99.52 | 99.33 | 99.13 |
| 7.23 | 1.08 | ND | ND | ND | 0.02 | 0.03 |
| 7.73 | 1.16 | ND | ND | 0.01 | 0.01 | 0.02 |
| 8.83 | 1.32 | 0.02 | 0.01 | 0.01 | 0.02 | 0.01 |
| 9.23 | 1.38 | ND | 0.02 | 0.02 | 0.03 | 0.04 |
| 9.62 | 1.44 | 0.03 | 0.00 | ND | ND | ND |
| 10.65 | 1.59 | ND | ND | ND | ND | ND |
| 11.44 | 1.71 | 0.01 | 0.01 | 0.03 | 0.07 | 0.07 |

ND = no peak detected

What is claimed is:

1. An aqueous solution comprising:
between about 2% (w/v) and about 12% (w/v) naloxone or a pharmaceutically acceptable salt thereof,
between about 2% (w/v) and about 25% (w/v) propylene glycol (PG), and
between about 0.2% (w/v) and about 1.8% (w/v) isotonicity agent;
wherein the solution comprises no more than about 2% (w/v) of alcohol, and
wherein the solution has a dynamic viscosity less than about 100 cP at 21° C.

2. The solution of claim 1, further comprising between about 0.05% and about 1% (w/v) of a stabilizing agent.

3. The solution of claim 2, wherein the solution comprises at least about 4% (w/v) naloxone or a pharmaceutically acceptable salt thereof.

4. The solution of claim 1, wherein the solution comprises at least about 4% (w/v) naloxone or a pharmaceutically acceptable salt thereof.

5. The solution of claim 2, wherein the PG is present in a concentration between about 5% and about 10% (w/v).

6. The solution of claim 2, wherein the PG is present in a concentration between about 15% and about 20% (w/v).

7. The solution of claim 3, wherein the osmolality is between about 350 mOsm and 2500 mOsm.

8. The solution of claim 7, wherein the stabilizing agent is present in a concentration between about 0.05% and about 0.15% (w/v).

9. The solution of claim 8, wherein the isotonicity agent is present in a concentration between about 0.6% and about 1% (w/v).

10. The solution of claim 8, further comprising an amount of acid or buffer sufficient to achieve a pH between about 3 and about 7.

11. The solution of claim 10, wherein:
the isotonicity agent is sodium chloride;
the stabilizing agent is disodium edetate; and
the acid is hydrochloric acid.

12. The solution of claim 11, wherein the solution comprises no detectable alcohol.

13. The solution of claim 12, further comprising between about 0.005% and about 0.015% (w/v) of a preservative.

14. The solution of claim 13, wherein the preservative is about 0.01% (w/v) benzalkonium chloride, and wherein the pH is between about 3.5 and about 5.5.

15. The solution of claim 11, wherein the solution does not contain any additional preservative, beyond the ingredients claimed, and wherein the pH is between about 3.5 and about 5.5.

16. The solution of claim 14, wherein the solution comprises:
about 4% (w/v) naloxone HCl;
between about 0.6% (w/v) and about 0.8% (w/v) NaCl;
about 5% (w/v) PG; and
between about 0.05% (w/v) and about 0.2% (w/v) disodium edetate.

17. The solution of claim 14, wherein the solution comprises:
about 4% (w/v) naloxone HCl;
between about 0.6% (w/v) and about 0.8% (w/v) NaCl;
about 10% (w/v) PG; and
between about 0.05% (w/v) and about 0.2% (w/v) disodium edetate.

18. The solution of claim 15, wherein the solution comprises:
about 4% (w/v) naloxone HCl;
between about 0.6% (w/v) and about 0.8% (w/v) NaCl;
about 5% (w/v) PG; and
between about 0.05% (w/v) and about 0.2% (w/v) disodium edetate.

19. The solution of claim 15, wherein the solution comprises:
about 4% (w/v) naloxone HCl;
between about 0.6% (w/v) and about 0.8% (w/v) NaCl;
about 10% (w/v) PG; and
between about 0.05% (w/v) and about 0.2% (w/v) disodium edetate.

20. The solution of claim 15, wherein the solution comprises:
about 4% (w/v) naloxone HCl;
between about 0.6% (w/v) and about 0.8% (w/v) NaCl;
about 15% (w/v) PG; and
between about 0.05% (w/v) and about 0.2% (w/v) disodium edetate.

21. The solution of claim 15, wherein the solution comprises:
about 4% (w/v) naloxone HCl;
between about 0.6% (w/v) and about 0.8% (w/v) NaCl;
about 20% (w/v) PG; and
between about 0.05% (w/v) and about 0.2% (w/v) disodium edetate.

22. The solution of claim 16, wherein the solution consists essentially of:
about 4% (w/v) naloxone HCl;
between about 0.6% (w/v) and about 0.8% (w/v) NaCl;
about 5% (w/v) PG; and
between about 0.05% (w/v) and about 0.2% (w/v) disodium edetate.

23. The solution of claim 17, wherein the solution consists essentially of:
about 4% (w/v) naloxone HCl;
between about 0.6% (w/v) and about 0.8% (w/v) NaCl;
about 10% (w/v) PG; and
between about 0.05% (w/v) and about 0.2% (w/v) disodium edetate.

24. The solution of claim 18, wherein the solution consists essentially of:
about 4% (w/v) naloxone HCl;
between about 0.6% (w/v) and about 0.8% (w/v) NaCl;
about 5% (w/v) PG; and
between about 0.05% (w/v) and about 0.2% (w/v) disodium edetate.

25. The solution of claim 19, wherein the solution consists essentially of:
about 4% (w/v) naloxone HCl;
between about 0.6% (w/v) and about 0.8% (w/v) NaCl;
about 10% (w/v) PG; and
between about 0.05% (w/v) and about 0.2% (w/v) disodium edetate.

26. The solution of claim 2, wherein the solution has a volume of about 80 μL to about 150 μL, and wherein the solution comprises:
about 4% (w/v) naloxone HCl;
about 0.5% (w/v) to about 1% (w/v) NaCl;
about 5% (w/v) to about 10% (w/v) PG; and
hydrochloric acid sufficient to achieve a pH of 3.5-5.5.

27. A pre-primed, single-use nasal spray device,
wherein the device comprises a reservoir, a piston, a swirl chamber, and a spray nozzle; and
wherein the reservoir contains a solution comprising:
at least about 2% (w/v) naloxone or a pharmaceutically acceptable salt thereof;
between about 2% (w/v) and about 15% (w/v) PG;
between about 0.2% and about 1.8% (w/v) of an isotonicity agent; and
no more than about 1% (w/v) of alcohol.

28. A mist, wherein the mist stands adjacent to a spray nozzle,
wherein the mist comprises droplets of a solution,
wherein no more than about 10% of the droplets have a diameter less than 10 μm as measured by laser diffraction at 3 cm and 6 cm from the spray nozzle,
wherein the solution has a dynamic viscosity less than 100 cP at 21° C., and
wherein the solution comprises:
at least about 2% (w/v) naloxone or a pharmaceutically acceptable salt thereof;
between about 2% (w/v) and about 15% (w/v) PG;
between about 0.2% and about 1.8% (w/v) of an isotonicity agent; and
no more than about 1% (w/v) of alcohol.

29. A method of treating opioid overdose in a patient in need thereof, the method comprising:
delivering a spray from a pre-primed, single-use nasal spray device into a nostril of the patient,
wherein a reservoir of the device contains a pharmaceutical solution comprising at least about 2% (w/v) naloxone or a pharmaceutically acceptable salt thereof, and between about 2% (w/v) and about 15% (w/v) PG.

30. The method of claim 29, further comprising storing the device at a temperature less than 0° C. for at least an hour.
